(19) United States
(12) Patent Application Publication
(10) Pub. No.: US 2022/0047504 A1
(43) Pub. Date: Feb. 17, 2022

(54) ORAL DISSOLVABLE FILM WITH PORES EXTENDING THERETHROUGH

(71) Applicant: Cure Pharmaceutical Holding Corp., Oxnard, CA (US)

(72) Inventors: Robert Davidson, Oxnard, CA (US); Steven Ruhl, Oxnard, CA (US); Vered Gigi, Oxnard, CA (US); Bhaumik Patel, Oxnard, CA (US); Jose Bernardo, Oxnard, CA (US); Maribel Espinoza, Oxnard, CA (US)

(73) Assignee: Cure Pharmaceutical Holding Corp., Oxnard, CA (US)

(21) Appl. No.: 17/331,731

(22) Filed: May 27, 2021

Related U.S. Application Data
(60) Provisional application No. 63/030,390, filed on May 27, 2020.

(51) Int. Cl. A61K 9/00

(52) U.S. Cl. CPC A61K 9/0056

(57) ABSTRACT

An oral dissolvable film, methods of making the oral dissolvable film, and methods of using the oral dissolvable film. The oral dissolvable film contains pores located therein.

### RELATED U.S. APPLICATION DATA

This application claims priority to U.S. provisional patent application No. 63/030,390 filed on May 27, 2020 the contents of which are incorporated by reference herein in its entirety.

### SUMMARY OF THE INVENTION

The present invention provides for an oral dissolvable film that includes a film matrix. The film matrix includes a film forming agent and one or more pharmaceutically acceptable excipients. The oral dissolvable film contains pores extending therethrough the oral dissolvable film.

The present invention also provides for an oral dissolvable film that includes a film matrix. The film matrix includes a film forming agent, one or more active pharmaceutical ingredients (APIs), and one or more pharmaceutically acceptable excipients. The oral dissolvable film contains pores extending therethrough the oral dissolvable film.

The present invention also provides for a method of manufacturing an oral dissolvable film containing pores extending therethrough. The method includes (a) forming or obtaining a cast slurry, or forming or obtaining a cured film; and (b) laser irradiating multiple times the cast slurry or laser drilling multiple times the cured film, to form pores extending therethrough.

The present invention also provides for a method of manufacturing an oral dissolvable film containing pores extending therethrough. The method includes (a) forming or obtaining a cured film; (b) laser drilling multiple times the cured film, to form pores extending therethrough;

(c) converting the cured film into desired dimensions; and (d) packaging.

The present invention also provides for a method of manufacturing an oral dissolvable film containing pores extending therethrough. The method includes (a) forming or obtaining a cast slurry; (b) laser irradiating multiple times the cast slurry, to form pores extending therethrough; (c) curing the cast slurry to form an oral dissolvable film with pores extending therethrough; (d) converting the cured film into desired dimensions; and (e) packaging.

The present invention also provides for a method of manufacturing an oral dissolvable film containing pores extending therethrough. The method includes (a) forming or obtaining a cast slurry; (b) mechanically piercing the cast slurry, to form pores extending therethrough; (c) curing the cast slurry to form an oral dissolvable film with pores extending therethrough; (d) converting the cured film into desired dimensions; and (e) packaging.

The present invention also provides for an oral dissolvable film that includes multiple film matrices, wherein at least one of the multiple film matrices contains the pores that extend therethrough. Each of the film matrices can independently include the same substances as the remaining film matrices e.g., each of the film matrices can independently be composed of the same substances as the remaining film matrices). Alternatively, any one or more of the film matrices can independently include different substances as the remaining film matrices (e.g., any one or more of the film matrices can independently be composed of different substances as the remaining film matrices). Either way, the pores can extend therethrough any one or more of the matrices.

When the oral dissolvable film includes a single film matrix, the method of manufacturing a cured film containing pores extending therethrough (as described herein) can also be a method of manufacturing the oral dissolvable film containing pores extending therethrough. Likewise, when the oral dissolvable film includes multiple film matrices, for those film matrices containing pores extending therethrough, the method of manufacturing a cured film containing pores extending therethrough (as described herein) can be employed. The multiple film matrices (post curing) can then be affixed to one another as desired. These multiple film matrices can independently include or omit pores, provided at least one matrix includes the pores.

With the methods of manufacturing an oral dissolvable film containing pores extending therethrough, as described herein, the cured film can be formed by: (a) forming or obtaining a slurry; (b) extruding the slurry and casting onto a substrate to form a cast slurry; and (c) curing the cast slurry to form a cured film,

### DETAILED DESCRIPTION OF THE INVENTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances. features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “excipient” refers to a pharmacologically inactive substance used in the pharmaceutical preparation of oral solid dosage forms (e.g., oral dissolvable film). An excipient is a substance formulated alongside the active pharmaceutical ingredient (API), and can include agents for stabilizing, bulking/filling, disintegrating, dissolving, flavoring, facilitating drug absorption, reducing viscosity, and/or enhancing solubility. Excipients can also be useful in the manufacturing process, to aid in the handling of the API, such as by facilitating powder flowability or non-stick properties, and/or aiding in vitro stability such as prevention or mitigation of degradation of the API over the expected shelf life. The selection of appropriate excipients also depends upon the route of administration and the dosage form, as well as the API and other factors. Pharmaceutical regulations and standards require that all ingredients in drug products, as well as their chemical decomposition products, be identified and shown to be safe. The Federation of International Pharmaceutical Excipients Council (IPEC), a pharmaceutical regulatory non-profit, develops, implements, and promotes global use of appropriate quality, safety, and functionality standards for pharmaceutical excipients and excipient delivery systems.

Excipients useful in the formulations described herein (e.g., slurry and ODE) include, e.g., binder, filler, preservative, sweetening agent, solvent, co-solvent, plasticizer, flavoring agent, taste masking agent, colorant, anti-caking agent, coating agent, emulsifier, solubilizing agent, lipid, humectant, thickening agent, lubricant, adsorbent, suspending agent, disintegrating agent, permeation enhancer, saliva stimulating agent, release modifier, adjuvant, fragrance, surfactant, pH adjusting agent, buffering agent, stabilizer, and antioxidant. Excipients that can be used in the formulation of oral dissolvable films are described in, e.g., Lachman, et al., “The Theory and Practice of Industrial Pharmacy,” 4^{th} Edition (2013); Sheskey et al., “Handbook of Pharmaceutical Excipients,” 9th Edition (2020); and Remington, “The Science and Practice of Pharmacy,” 22nd Edition (2015), All excipients used in the formulation of the oral dissolvable film described herein, in the desired amounts, should preferably be approved by the FDA for use in oral pharmaceutical dosage forms. See, e.g., the FDA Inactive Ingredient Database (IID), https://www.accessdata.fda.gov/scripts/cder/iig/index.cfm (accessed Apr. 15, 2021). Additionally, the excipients will preferably be commercially available in acceptable grade, physiologically inert, and physically and chemically stable by themselves, as well as in combination with the desired API.

Reference can be made to the slurry (and the resulting oral dissolvable film) as containing (or as being manufactured from, or as being formed from) various substances, such as the active ingredient and multiple excipients (e.g., binder, filler, flavoring agent, plasticizer, sweetening agent, coloring agent, preservative, and/or solvent). As described herein, within the context of the present invention, it is appreciated that those of skill in the art understand and agree that reference to the slurry (and the resulting oral dissolvable film) as containing the active ingredient and excipients is acceptable and appropriate. This is so, even though those substances may no longer necessarily exist in the same state as when introduced into the slurry, as specifically indicated. Likewise, within the context of the present invention, reference can also be made to the slurry (and the resulting oral dissolvable film) as being manufactured from (or as being formed from) the active ingredient and excipients, as specifically indicated. It is appreciated that those of skill in the art understand and agree that each of the above characterizations of the slurry (and the resulting oral dissolvable film) are acceptable and appropriate.

The term “inactive ingredient” (and equivalent terms such as “inactive component”) refers to a substance that is pharmacologically and biologically inactive. The inactive ingredients are usually called excipients in pharmaceutical contexts.

The term “slurry” refers to a mixture of solids that is dispersed, suspended, solubilized, and/or dissolved in liquid. Together, the solids and liquid will include those substances used to manufacture the oral dissolvable film. The solid substances employed in the manufacture of the oral dissolvable film can essentially be dissolved in the liquid, can essentially be suspended in the liquid, can essentially be dispersed in the liquid, can essentially be solubilized in the liquid, or a combination thereof. An oral dissolvable film can be formed by curing a cast slurry. The curing can be carried out, e.g., at an elevated temperature, for a period of time. In doing so, a significant amount of the solvent (e.g., water) will be removed. The remaining water will contribute to the moisture content in the oral dissolvable film (in addition to any moisture from the surrounding environment that is picked up by any hygroscopic polymers employed).

The term “binder” (and equivalent terms such as “binding agent”) refers to a substance, typically a polymer, used in the pharmaceutical preparation of oral solid dosage forms (e.g., oral dissolvable film) to hold the ingredients together. Binders ensure that the oral dissolvable films can be formed with the requisite mechanical strength. The binders also provide the requisite volume to low amount of active present in soluble films. The presence of the binder can also facilitate the formation of the cured film. As such, the binder includes those substances, which when present in the cast slurry and upon curing, will effectively provide for a cured film. in designing of an oral dissolvable film formulation, consideration should be given to the target product and drug release profile. A primary component of an oral film is the binder, which may a polymer blend. Selection of the binder may be guided by the desired strength and stability of the oral dissolvable film, as well as mucoadhesiveness, pliability, dissolution rate, and moisture content. The binder may also be referred to as a “film forming agent,” or more specifically a “film forming polymer” (or equivalent terms, such as “strip-forming polymer” and “mucoadhesive polymer”) when it is a polymer. Polymeric binders (film forming agents) can be natural or a synthetic. Employing a binder can allow for, and promote, the formation of a “film matrix” (also referred to as a polymeric matrix), A film matrix is typically obtained by curing the cast slurry, which contains the hinder(s). Examples of binders for use in an ODF described herein include polyacrylic acid (PAA) (alternatively referred to as poly(acrylic acid) or Carbomer®); 1-polyacrylic acid; methyl methacrylate copolymer; carboxyvinyl polymer; polyethylene glycol (PEG) (alternatively referred to as polyethylene oxide or PEO); acacia; agar; alginic acid (alternatively referred to as algin); sodium alginate (Na alginate); calcium carbonate; calcium lactate; carboxymethyl cellulose (CMC) (alternatively referred to as cellulose gum or carboxy methylcellulose or carboxymethylcellulose); carrageenan; cellulose acetate; chitosan; copovidone; starch (e.g., corn starch or pregelatinized starch); cottonseed oil; dextrates; dextrin; dextrose (alternatively referred to as corn sugar and D-glucose); ethylcellulose; (alternatively referred to as ethyl cellulose); gelatin; guar gum; hydroxyethyl cellulose (HEC); hydroxyethyl methyl cellulose (MEMO); hydroxypropyl methylcellulose (HPMC) (alternatively referred to as hydroxypropyl methyl cellulose, hypromellose, or INN) (e.g., Vivapharm® HPMC E3, Methocel™ HPMC K3, Vivapharm® HPMC E5, Vivapharm® HPMC E15, or Methocel™ E15); hydroxypropyl cellulose (HPC); low substituted hydroxypropyl cellulose (L-HPC); hydroxypropyl starch; inulin; lactose; maltodextrin; maltose; methylcellulose (MC) (e.g., Methocel® A15); microcrystalline cellulose (MCC) (e.g., Avicel® PH-101); pectin; poloxamer (e.g., Plutonic®, Kolliphor®, and Synperonic®); polycarbophil; polydextrose; polymethacrylates; polyvinyl alcohol (PVA) (alternatively referred to as polyvinyl alcohol), polyvinylalcohol or PVOH or PVAI); polyvinylpyrrolidone (PVP) (alternatively referred to as polyvidone or povidone) (e.g., Kollidon® K90, Kollidon® 12 PF, Kollidon® 17 PF, Kollidon® 25 PF, or Kollidon® 30 PF); pullulan; sodium carboxymethylcellulose (CMC-Na) (alternatively. referred to as sodium carboxymethyl cellulose) (e.g., Cekol® 30); sucrose; sunflower oil; zein; vinylpyrrolidone-vinyl acetate copolymer (e,g., Kollidon® VA64); polyvinyl acetate/polyvinylpyrrolidone (e.g., Kollidon® SR); polyvinyl alcohol-polyethylene glycol copolymer and polyvinyl alcohol (PVA) (e.g., Kollicoat® Protect); polyvinyl alcohol/polyethylene glycol graft copolymer (e.g., Kollicoat® IR); polyvinyl caprolactampolyvinyl acetate-polyethylene glycol graft co-polymer (e,g., Soluplus®); poly(ethyl acrylate-co-methyl methacrylate-co-trimethylammonioethyl methacrylate chloride) (e.g., Eudragit® RL100); amino methacrylate copolymer (e.g., Eudragit® E PO); and xanthan gum.

The term “filler” (and equivalent terms such as “diluent” and “bulking agent”) refers to substances used in the pharmaceutical preparation of oral solid dosage forms (e.g., oral dissolvable film) to add bulk to the pharmaceutical dosage form, improving the consistency in dose metering and/or making the active ingredient easier for consumer to take. Fillers can also help with the manufacturing and stabilization of these products. Fillers can also bind and stabilize the dosage form. They are employed in the manufacture of an ODF to increase weight/mass and/or to improve content uniformity. Fillers can provide properties such as improved cohesion and/or to promote flow. Examples of fillers for use in an ODF described herein include anhydrous lactose, calcium carbonate, calcium lactate, calcium phosphate (dibasic anhydrous, dibasic dihydrate, or tribasic), calcium silicate, calcium sulfate, cellulose (powdered or silicified microcrystalline), cellulose acetate, corn starch and pregelatinized starch, dextrates, dextrin, dextrose, erythritol, ethylcellulose, fructose, fumaric acid, glucose, glyceryl pahnitostearate, glycine, hydrolyzed starch, lactose, lactose monohydrate, isomalt, kaolin, lactitol, magnesium carbonate, magnesium oxide, maltodextrin, maltose, mannitol, medium-chain triglycerides, microcrystalline cellulose (MCC), partially pregelatinized starches, plant cellulose, polydextrose, polymethacrylates, simethicone, sodium alginate, sodium chloride, sorbitol, pregelatinized starch, sterilizable maize, sucrose, sugar spheres, sulfobutylether cyclodextrin, talc, tragacanth, trehalose, and xylitol.

The term “preservative” refers to a substance used in the pharmaceutical preparation of oral solid dosage forms (e.g., oral dissolvable film), to prevent or mitigate microbial growth or by undesirable chemical changes. In general, preservation is implemented in two modes, chemical and physical. Examples of preservatives for use in an ODF described herein include ethanol, benzoic acid, benzyl alcohol, butylated hydroxy anisole (BHA), butylated hydroxytoluene (BHT), butylparaben, calcium acetate, calcium chloride, calcium lactate, cetylpyridinium chloride, chlorhexidine, chlorobutanol, citric acid monohydrate, ethylparaben, glycerin, lactic acid, methylparaben, parabens, potassium benzoate, potassium sorbate, propyl gallate, propylene glycol, propytparaben, propylparaben sodium, sodium acetate, sodium benzoate, sodium lactate, sodium propionate, sorbic acid, sulfohutyl ether B-cyclodextrin, edetic acid, xanthan, and xylitol.

The term “sweetening agent” (and equivalent terms such as “sweetener”) refers to a substance used in the pharmaceutical preparation of oral solid dosage forms (e.g., oral dissolvable film), to impart a sweet taste like that of sugar. The sweetener can be artificial or naturally occurring. Examples of sweetening agents for use in an OMF described herein include sugar, acesulfame salts (e.g., acesulfame potassium (ACE-K)), alitame, aspartame, dextrose, erythritol, fructose, glycerin, isomalt, lactitol, advantame, monk fruit extract (mogrosides), glucose, galactose, maltitol, maltose, mannitol, monk fruit extract, neohesperidin dihydrochal cone, neotame, saccharin, saccharin salts (e.g., saccharin sodium), sodium cyclamate, sorbitol, stevia, stevioside, rebaudioside A, sucralose, sucrose, tagatose, thaumatin, trehalose, licorice extract, and xylitol.

The term “solvent” refers to a substance that is used in the pharmaceutical preparation of an oral dissolvable film, to dissolve the active pharmaceutical ingredient (API) and/or excipients. A solvent can be employed to form a slurry. For most manufacturing methods, solvents improve solubility of the active ingredient within the film forming matrix. Solvents may be chosen based on the active ingredient's solubility therein. Preferred solvents include volatile class 3 residual solvents such as ethanol and acetone and non-volatile solvents such as water. In some embodiments, the solvent is at least one of ethanol and water. Upon curing of a slurry to provide an oral dissolvable film, a significant portion of the solvent will typically be removed, leaving behind a remaining (or residual) portion of the solvent.

The term “co-solvent” refers to a substance that is used in the pharmaceutical preparation of an oral dissolvable film, to assist the solvent in dissolving the active pharmaceutical ingredient (API) and/or excipients, to form a slurry. Examples of co-solvents for use in an ODF described herein include almond oil, castor oil, corn oil, cottonseed oil, ethanol, glycerin, olive oil, polyethylene glycol, polyoxy 35 castor oil, propylene glycol, safflower oil, sesame oil, soybean oil, sunflower oil, and ethanol. Upon curing of a slurry to provide an oral dissolvable film, a portion of the co-solvent may be removed.

The term “plasticizer” refers to a substance that, when added to polymer(s), makes the polymer more pliable and softer, enhancing the flexibility and plasticity of the films. They can be added to reduce the glass transition temperature to reduce the risk of thermally destabilizing the active ingredient and/or excipients. The plasticizer is believed to permeate the polymer structure, disrupting intermolecular hydrogen bonding, and permanently lowers intermolecular attractions. Plasticizers can be used to allow initial film forming, to reduce the brittleness, and improve the processability and flexibility of the resulting film, thereby avoiding cracking, e.g., during the curing process. Plasticizers can be used to improve elasticity of the oral dissolvable film which can be important for manufacturing scale-up. Plasticizers can also play a role when combined with certain polymers in the overall dissolution rate of the film. Examples of plasticizers for use in an ODF described herein include castor oil, glycerin, glycerol monostearate, D, hypromellose phthalate, mannitol, mineral oil and, palmitic acid, polyethylene glycol, polyvinyl acetate phthalate, propylene glycol, pyrrolidone, sorbitol, stearic acid, triacetin, tributyl citrate, triethyl citrate, water, glycerin fatty acid esters, sucrose fatty acid esters, lecithin, enzyme modified lecithin, polysorbates, sorbitan fatty acid esters, maltitol, xylitol, polyethylene glycol (PEG), hydrogenated starch syrup, starch syrup, and glycerol oleate.

The term “flavoring agent” (and equivalent terms such as “flavoring substance,” “flavor,” “flavoring,” and “flavorant”) refers to a substance used in the pharmaceutical preparation of an oral dissolvable film, to impart a flavor, e.g., to improve the attractiveness and acceptance by the subject. The basic taste sensations are salty, sweet, bitter, sour, and umami. Flavors may be chosen from natural and synthetic flavorings. An illustrative list of such agents includes volatile oils, synthetic flavor oils, flavoring aromatics, oils, liquids, oleoresins or extracts derived from plants, leaves, flowers, fruits, stems and combinations thereof. The flavoring agent can be available as a solid (e.g., powder), as a liquid (e.g., oil), or a combination thereof. Additionally, the flavoring agent for use in the ODF can include any one or more of a natural flavoring substance, a nature-identical flavoring substance, and an artificial flavoring substance. Examples of flavoring agents for use in an ODF described herein include allspice, anise, n-butyl lactate, cardamom, cherry, confectioner's sugar, cinnamon, clove, ethyl vanillin, ethyl cellulose, eugenol, ginger, gingermint, lemon, lemongrass, levomenthol, lime, alalic acid, maltol, menthol, mint, nutmeg, orange, peppermint, phosphoric acid, propionic acid, sodium acetate, sodium lactate, spearmint, tangerine, tartaric acid, thymol, triethyl citrate, vanillin, vanilla cream, watermint, wintergreen, mountain berry, and grape.

The term “natural flavoring substance” refers to a flavoring substance obtained from plant or animal raw materials, by physical, microbiological, or enzymatic processes. They can be either used in their natural state or processed for human consumption, but cannot contain any nature-identical or artificial flavoring substances,

The term “nature-identical flavoring substance” refers to a flavoring substance obtained by synthesis or isolated through chemical processes, which is chemically and organoleptically identical to flavoring substances naturally present in products intended for human consumption. They cannot contain any artificial flavoring substances.

The term “artificial flavoring substance” refers to a flavoring substance that is not identified in a natural product intended for human consumption, whether or not the product is processed. These are typically produced by fractional distillation and additional chemical manipulation of naturally sourced chemicals, crude oil, or coal tar. Although they are chemically different, in sensory characteristics they are the same as natural ones. Most artificial flavors are specific and often complex mixtures of singular naturally occurring flavor compounds combined. together to either imitate or enhance a natural flavor. These mixtures are formulated by flavorists to give a food product a unique flavor and to maintain flavor consistency between different product batches or after recipe changes. The list of known artificial flavoring agents includes thousands of molecular compounds, and the flavor chemist (flavorist) can often mix these together to produce many of the common flavors. Many of these artificial flavorants consist of esters, which are often described as being “sweet” or “fruity”.

The term “taste masking agent” refers to a substance used in the pharmaceutical preparation of an oral dissolvable film, to mask the unpleasant taste of a substance present in the formulation, to improve the attractiveness and acceptance by the subject. The taste masking agent can specifically refer to a substance used to mask the bitter or unpleasant taste of the active ingredient. Examples of taste masking agents for use in an ODF described herein include alginic acid, erythritol, and glyceryl palmitostearate, and monoammonium glycyrrhizinate (MAG). With the oral dissolvable films described herein, in addition to masking the taste of any unpleasant or bitter tasting substances (e.g., the active ingredient) present in the oral dissolvable film, the taste masking agent can optionally also impart a pleasant flavor. in such embodiments, the same substance can serve as both a flavoring agent and as a taste masking agent.

The term “colorant” (and equivalent terms such as “coloring agent”) refers to substance used in the pharmaceutical preparation of an oral dissolvable film, to change the color of the slurry and/or oral dissolvable film. The colorant is a dye, pigment, or substance that imparts color when it is added to a slurry. Colorants work by absorbing varying amounts of light at different wavelengths (or frequencies) of its spectrum, transmitting (if translucent) or reflecting the remaining light in straight lines or scattered. Color consistency can be significant, as it allows easy identification of a medication to the subject. Furthermore, colors often improve the aesthetic look and feel of medications. By increasing these organoleptic properties, a subject is more likely to adhere to their schedule and therapeutic objectives will also have a better outcome for the subject. The colorant can include, e.g., FD&C colors (e.g., FD&C red, FD&C yellow, FD&C blue, FD&C green), D&C colors, or a combination thereof. Examples of colorants for use in an ODF described herein include FD&C Red No. 3, FD&C Red No. 40, FD&C Yellow No. 5, FD&C Yellow No. 6, FD&C Green No. 3, FD&C Blue No. 1, and FD&C Blue No. 2.

The term “anti-caking, agent” (and equivalent terms such as “anti-tacking agent”) refers to a substance used in the pharmaceutical preparation of oral solid dosage forms (e.g., oral dissolvable film), to prevent or mitigate the occurrence of the formation of lumps (caking) of powdered or granulated materials. Use of the anti-tacking agent can result in the ease of flowability of the solid powders used to form the slurry. Crystalline solids often cake by formation of liquid bridge and subsequent fusion of microcrystals. Amorphous materials can cake by glass transitions and changes in viscosity. Examples of anti-caking agents for use in an ODF described herein include calcium silicate, tribasic calcium phosphate, colloidal silicon dioxide, hydrophobic colloidal silica, magnesium oxide, magnesium silicate, magnesium trisilicate, and talc, mannitol, and starch.

The term “coating agent” refers to a substance that is applied to a solid or powder particle, to sufficiently coat the particle. The thickness of such a coating is usually less than 100 μm. The motivation for coating a particle ranges from improving the stability (light protection, moisture and gas barrier) to increasing the flowability to thereby make it easier to process. Examples of coating agents for use in an ODF described herein include calcium carbonate, carboxymethylcellulose calcium, carboxymethylcellulose sodium, carnauba wax, cellulose acetate, (CAP), chitosan, ethylcellulose, fructose, gelatin, glycerin, glyceryl behenate, glyceryl palmitostearate, hydroxyethyl cellulose, hydroxypropyl cellulose, hypromellose, hypromellose phthalate, iso alt, glucose, maltitol, maltodextrin, methylcellulose, microcrystalline wax, poloxamer, polydextrose, polyethylene glycol, poly-DL-(lactic acid), polyvinyl acetate phthalate, polyvinyl alcohol, povidone, sucrose, titanium oxide, tributyl citrate, triethyl citrate, vanillin, xylitol, and zein.

The term “emulsifier” (and equivalent terms such as “emulsifying agent”) refers to a substance capable of forming or promoting an emulsion. in particular reference to the oral dissolvable films described herein, the emulsifier can promote the separation of phases (e.g., aqueous and lipids), while allowing them to be mixed. Examples of emulsifiers for use in an ODF described herein include acacia, cholesterol, glycerin, glyceryl monostearate, hydroxypropyl cellulose, lecithin, methylcellulose, mineral oil and, monobasic sodium phosphate, monoethanolamine, oleic acid, polyethylene glycol, poloxamer, polyoxyethylene alkyl ethers, polyoxyethylene castor oil derivatives, polyoxyethylene sorbitan fatty acid esters, polyoxyethylene stearates, polysorbate (e.g., polysorbate 20, polysorbate 40, polysorbate 60, polysorbate 80, etc.), propylene glycol, propylene glycol alginate, sodium lawyl sulfate, sorbitan esters, and stearic acid.

The term “solubilizing agent” (and equivalent terms such as “solubilizer”) refers to a substance used in the pharmaceutical preparation ofan oral dissolvable film, to increase the solubility and/or bioavailability of the API. A solubilizing agent can act as a surfactant and increases the solubility of one agent in another. A substance that would not normally dissolve in a particular solution may be able to dissolve with the use of a solubilizing agent. Examples of solubilizing agents for use in an ODF described herein include cyclodextrins, glycerin monostearate, hydroxpropyl betadex, hypromellose, inulin, lecithin, meglumine, phospholipids, poloxamer, polyoxyethylene alkyl ethers, polyoxyethylene castor oil derivatives, polyoxyethylene sorbitan fatty acid esters, polyoxyethylene stearates, polyoxylglycerides, povidone, pyrrolidone, sorbitan esters, starch, stearic acid, sulfobutylether β-cyclodextrin, tricaprylin, triolein, and vitamin E polyethylene glycol succinate, N-acetylated amino-acid derivative, ethoxylated sorbitan, mono and diglycerides.

The term “emulsion”m refers to a mixture of two or more liquids that are normally immiscible (unmixable or unblendable) owing to liquid-liquid phase separation. Two liquids can form different types of emulsions. As an example, oil and water can form, first, an oil-in-water emulsion, in which the oil is the dispersed phase, and water is the continuous phase. Second, they can form a water-in-oil emulsion, in which water is the dispersed phase and oil is the continuous phase. Multiple emulsions are also possible, including a “water-in-oil-in-water” emulsion and an “oil-in-water-in-oil” emulsion. Emulsions, being liquids, do not exhibit a static internal structure. The droplets dispersed in the continuous phase (sometimes referred to as the “dispersion medium”) are usually assumed to be statistically distributed to produce roughly spherical droplets. When molecules are ordered during liquid-liquid phase separation, they form liquid crystals rather than emulsions. Lipids, used by all living organisms, are one example of molecules able to form either emulsions (e.g.: spherical micelles; Lipoproteins) or liquid crystals (lipid bilayer membranes). The droplets may be amorphous, liquid-crystalline, or any mixture thereof. The diameters of the droplets constituting the dispersed phase usually range from approximately 10 nm to 100 μm; i.e., the droplets may exceed the usual size limits for colloidal particles. An emulsion is termed an oil/water (o/w) emulsion if the dispersed phase is an organic material, and the continuous phase is water or an aqueous solution and is termed water/oil (w/o) if the dispersed phase is water or an aqueous solution and the continuous phase is an organic liquid (an “oil”).

Two special classes of emulsions—microemulsions and nanoemulsions, with droplet sizes below 100 nm—appear translucent. This property is due to the fact that light waves are scattered by the droplets only if their sizes exceed about one-quarter of the wavelength of the incident light. Since the visible spectrum of light is composed of wavelengths between 390 and 750 nanometers (nm), if the droplet sizes in the emulsion are below about 100 nm, the light can penetrate through the emulsion without being scattered. Due to their similarity in appearance, translucent. nanoemulsions and microemulsions are frequently confused. Unlike translucent nanoemulsions, which require specialized equipment to be produced, microemulsions may be spontaneously formed by “solubilizing” oil molecules with a mixture of surfactants, co-surfactants, and co-solvents. The required surfactant concentration in a microemulsion is, however, several times higher than that in a translucent nanoemulsion, and significantly exceeds the concentration of the dispersed phase. Because of many undesirable side-effects caused by surfactants, their presence is often considered disadvantageous or prohibitive in many applications. In addition, the stability of a microemulsion may be compromised by dilution, by heating, or by changing pH levels.

The term “lipid” refers to a group of naturally occurring molecules that include fats, waxes, sterols, fat-soluble vitamins (such as vitamins A, D, E, and K), monoglycerides, diglycerides, triglycerides, phospholipids, and others. “Lipid” may also refer to ethoxylated fatty alcohols such as oleth-10 and laureth-10 and mixtures of ethoxylated mono and diglycerides such as PEG-16 macadamia glycerides and PEG-10 sunflower glycerides. The compounds are hydrophobic or amphiphilic small molecules. The amphiphilic nature of some lipids allows them to form structures such as vesicles, liposomes, or membranes in an aqueous environment. Biological lipids originate entirely or in part from two distinct types of biochemical subunits or “building-blocks”: ketoacyl and isoprene groups. Using this approach, lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits). Although the term lipid is sometimes used as a synonym for fats, fats are a subgroup of lipids called triglycerides. Lipids also encompass molecules such as fatty acids and their derivatives (including tri-, di-, monoglycerides, and phospholipids), as well as other sterol-containing metabolites such as cholesterol. Examples of lipids for use in an ODF described herein include almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, Shea butter, soybean oil, sunflower oil, sunflower lecithin, walnut oil, and watermelon seed oil, vitamin E, mono and diglycerides, propylene glycol, polyethylene glycol, Kolliphor® RH, and Kolliphor® EL.

The term “humectant” refers to a substance used in the pharmaceutical preparation of an oral dissolvable film, to keep the slurry and/or oral dissolvable film moist. A humectant attracts and retains the moisture in the air nearby via absorption, drawing the water vapor into or beneath the oral dissolvable film's surface. This is the opposite use of a hygroscopic material where it is used as a desiccant used to draw moisture away. Humectants can be used in oral dissolvable films to help solubilize active ingredients, increasing the active ingredients' ability to penetrate a mucosal surface, or its activity time. Examples of humectants for use in an ODF described herein include glycerin, polydextrose, propylene glycol, sodium lactate, sorbitol, trehalose, triacetin, xylitol, sodium chloride, and polyvinylpyrrolidone.

The term “thickening agent” (and equivalent terms such as “gelling agent” and “viscosity increasing agent”) refers to substances used in the pharmaceutical preparation of oral dissolvable films, to improve the viscosity and consistency of the slurry before casting. Active ingredient content uniformity is often a requirement for dosage forms, particularly those containing low dose highly potent active ingredients. To uniquely meet this requirement, oral dissolvable film formulations can contain uniform dispersions of active ingredient throughout the manufacturing process. Examples of thickening agents for use in an ODF described herein include acacia, agar, alginic acid, calcium alginate, carboxymethylcellulose calcium, carboxymethylcellulose sodium, carrageenan, ceratonia, chitosan, cyclomethicone, ethylcellulose, gelatin, glycerin, guar gum, hydrogenated vegetable oil, hydroxyethy cellulose, hydroxypropyl cellulose, hydroxypropyl starch, hypromellose, locust bean gum, maltodextrin, methylcellulose, pectin, polydextrose, polyethylene glycol, polyvinyl alcohol, potassium chloride, potassium alginate, povidone, propylene glycol alginate, sodium alginate, sodium chloride, starch, sucrose, sulfobutylether β-cyclodextrin, and xanthan gum.

The term “lubricant” (and equivalent terms such as “glidant”) refers to a substance used in the pharmaceutical preparation of oral solid dosage forms (e.g., oral dissolvable film) to improve processing characteristics. For example, the lubricant can enhance flow of the slurry by reducing interparticulate friction. The lubricant is typically added to a powder to improve its flowability. A lubricant will typically only work at a certain range of concentrations. Above a certain concentration, the lubricant will function to inhibit flowability. Examples of lubricants for use in an OMF described herein include ascorbyl palmitate, calcium palmitate, castor oil, fumed silica (colloidal silicon dioxide), glycerin monostearate, glyceryl palmitostearate, hydrogenated castor oil, hydrogenated vegetable oil (e.g., Sterotex®, Lubritab®, and Cutina®), light mineral oil, magnesium stearate, medium-chain triglycerides, mineral oil, palmitic acid, partial fatty acid esters of sugars, polyethylene glycol fatty acid esters, polyethylene glycol fatty alcohol ethers, polyethylene glycol sorbitan fatty acid esters, 2-ethoxy ethanol, ethyl alcohol, poloxamer, polyethylene glycol, sodium benzoate, sodium chloride, starch, sucrose esters, and talc.

The term “adsorbent” refers to a substance used to prevent or mitigate the occurrence of another substance from undergoing sorption (process in which one substance takes up or holds another, by either absorption or adsorption). Typically, the adsorbent is used to prevent or mitigate another substance from taking up water or moisture. Examples of adsorbents for use in an ODF described herein include aluminum hydroxide, aluminum oxide, aluminum phosphate, attapulgite, bentonite, powdered cellulose, colloidal silicon dioxide, magnesium aluminum silicate, microcrystalline cellulose, pectin, polycarbophil, and talc.

The term “suspending agent” refers to a substance that helps another substance (e.g., active pharmaceutical ingredient) to stay suspended in the formulation (e.g., slurry) and to prevent or mitigate the occurrence of caking at the bottom of the container. One of the properties of a well-formulated suspension is that it can be easily re-suspended by the use of moderate agitation or blending. Typically, the suspending agent will help other substances to stay suspended in the slurry, prior to the curing. In doing so, the substances are held in the slurry by the suspending agent, and do not settle at the bottom to any appreciable degree. Examples of suspending agents for use in an ODF described herein include acacia, agar, alginic acid, bentonite, calcium stearate, carbomers, carboxymethylcellulose calcium, carboxymethylcellulose sodium, carrageenan, powdered cellulose, cellulose (microcrystalline and carboxymethylcellulose sodium), colloidal silicon dioxide, dextrin, gelatin, guar gum, hydroxyethyl cellulose, hydroxypropyl cellulose, hypromellose, kaolin, magnesium aluminum silicate maltitol solution, medium-chain triglycerides, methylcellulose, microcrystalline cellulose, phospholipids, polycarbophil, polyethylene glycol, polyoxyethylene sorbitan fatty acid esters, potassium alginate, povidone, propylene glycol alginate, sesame oil, sodium alginate, sodium starch glycolate, sorbitan esters, sucrose, tragacanth, vitamin E polyethylene glycol succinate, and xanthan gum.

The term “disintegrating agent” (and equivalent terms such as “disintegrator” and “disintegrant”) refers to a substance used in the pharmaceutical preparation of oral solid dosage forms (e.g., oral dissolvable film or orally disintegrating tablet), that helps the dosage form to disintegrate and release the active ingredient on contact with moisture. The disintegrant is employed in the manufacture of an ODF to promote its rapid disintegration or break down into small particles after administration for facilitating rapid dissolution into bodily fluid. Examples of disintegrating agents for use in an ODF described herein include alginic acid, Amberlite™, calcium alginate, carboxymethylcellulose calcium, carboxymethylcellulose sodium, powdered cellulose, chitosan, colloidal silicon dioxide, coin starch and pregelatinized starch, croscarmellose sodium, crospovidone, glycine, guar gum, hydroxypropyl cellulose, microcrystalline cellulose, polacrilin potassium, povidone, sodium starch glycolate, pregelatinized starch, and low substituted HPMC.

The term “permeation enhancer” refers to a substance used in the pharmaceutical preparation of an oral dissolvable film, to increase the delivery of the active ingredient, when administered in vivo (e.g, orally), resulting in an increased absorption of the active ingredient.

The active ingredient can be delivered across the desired body surface, e.g., oral mucosa, such as buccal, sublingual, mucosa, or gingival; or an intestinal surface. Examples of permeation enhancers for use in an ODF described herein include anionic surfactants (e.g., sodium lauryl sulfate, sodium laurate, Laureth-9, sodium dodecyl sulfate (SDS), dioctyl sodium sulfosuccinate), nonionic surfactants (polyoxyethylene-9-lauryl ethe (PLIC;), Tween® 80, nonylphenoxypolyoxyethylene (NPPOE), polysorbate, sodium glycocholate), cationic surfactants (e.g., cetylpyridinium chloride, chitosan, trimethyl chitosan, poly-L-arginine, L-lysine), fatty acids or derivatives thereof (e.g., oleic acid caprylic acid, mono(di)glycerides, lauric acid, linoleic acid, acyicholines, acylcarnitine, sodium caprate, and oleic acid), and polyols (e.g., propylene glycol, polyethylene glycol, glycerol, or propanediol).

The present invention relates to a dissolvable film that can be used to administer a desired predetermined substance, referred to herein as an “active pharmaceutical ingredient” (API) (and equivalent terms such as “active ingredient,” etc.), at an amount sufficient or effective to (1) obtain a desired result, such as the treatment of the subject, to (2) obtain a desired level of API in the subject (as evidenced by, e.g., plasma levels of the API), and/or (3) obtain a desired level of API active metabolite in the subject (as evidenced by, e.g., plasma levels of the API active metabolite).

The term “active pharmaceutical ingredient” or “API” (and equivalent terms such as “active ingredient,” “medicant,” “medicament,” “bioactive,” and “active”) refers to a substance for use in the treatment of a disease or disorder. Dietary supplements, vitamins, functional foods (e.g., ginger, green tea, lutein, garlic, lycopene, capsaicin, and the like) are also included in this term. The active pharmaceutical ingredient is pharmacologically and biologically active. Standard references such as, e.g., The Physician's Desk Reference, 2018 Edition; The Merck Index, 15th Edition (2013); and United States Pharmacopeia (USP) (2018) provide a description of specific active pharmaceutical ingredients, and pharmaceutically acceptable salts thereof, suitable for use with the dissolvable films described herein.

The term “saliva stimulating agent” (and equivalent terms such as “salivary stimulant” and “acidulant”) refers to a substance used to increase the production of saliva, thereby increasing salivary flow rate. Examples of saliva stimulating agents for use in an ODF described herein include organic acids (e.g., ascorbic acid, citric acid, fumaric acid, tartaric acid, and malic acid), parasympathomimetic drugs (e.g., choline esters such as pilocarpine hydrochloride and cholinesterase inhibitors), physostigmine, and other substances (e.g., xylitol, xylitol/sorbitol, and nicotinamide).

The term “release modifier” refers to a substance used to modify the release of the active ingredient from an oral solid dosage form (e.g., ODF) and/or is used to modify the absorption of the active ingredient when the oral solid dosage form is orally administered to the subject. The drug release can be contrasted to an immediate release (IR), and includes, e.g., an extended release (XR), sustained release (SR), or delayed release (DR).

The term “adjuvant” refers to a substance used to modify (e.g., increase) the effect or efficacy of the active ingredient present in an oral solid dosage form (e.g., ODF). The adjuvant can be, e.g., a pharmacological agent or immunological agent.

The term “fragrance” (and equivalent terms such as “fragrant,” “odorant,” or “aroma compound”) refers to a substance used to impart a desired smell, scent, or odor to a formulation (e.g., slurry or ODF).

The term “surfactant” refers to a substance used to lower the surface tension (or interfacial tension) between two liquids, between a gas and a liquid, or between a liquid and a solid. Surfactants may act as detergents, wetting agents, emulsifiers, foaming agents, or dispersants. The surfactant can be anionic, cationic, zwitterionic, or non-ionic.

The term “pH adjusting agent” refers to a substance used to change the pH of an aqueous solution (e.g., slurry). For example, the pH adjusting agent can be an acid, such that when added to an aqueous solution (e.g., slurry), it will decrease the pH. Alternatively, the pH adjusting agent can be a base, such that when added to an aqueous solution (e.g., slurry), it will increase the pH. The base can be an organic base (e.g., sodium bicarbonate), an inorganic base sodium hydroxide), or combination thereof. Likewise, the acid can be an inorganic acid (e.g., hydrochloric acid), an organic acid (e.g., citric acid, malic acid, tartaric acid, etc.), or a combination thereof.

The term “buffering agent” refers to a weak acid or weak base used to maintain the pH (e.g., acidity or basicity) of a solution (e.g., slurry) near a chosen value after the addition of is another acid or base. That is, the function of a buffering agent is to prevent or mitigate the occurrence of a rapid change in pH when acids or bases are added to the solution (e.g., slurry). Buffering agents have variable properties—some are more soluble than others; some are acidic while others are basic. The acid can be an organic acid, mineral acid, or combination thereof. Likewise, the base can be an organic base, inorganic base, or combination thereof.

The term “stabilizer” refers to a substance used to prevent or mitigate the occurrence of degradation of any one of more substances present in a formulation (e.g., the slurry and/or oral dissolvable film). This would include preventing or mitigating degradation of the active ingredient, as well as any of the inactive ingredients or excipients.

The term “antioxidant” refers to a substance used to inhibit, prevent, or mitigate the occurrence of oxidation of any one of more substances present in a formulation (e.g., the slurry and/or oral dissolvable film). This would include inhibiting, preventing, or mitigating oxidation of the active ingredient, as well as any of the inactive ingredients or excipients. Examples of antioxidants for use in an ODF described herein include ascorbic acid (vitamin C), vitamin A, α-tocopherol (vitamin F), beta-carotene, glutathione, ubiquinol (coenzyme Q), and selenium.

The term “oral dissolvable film” or “ODF” (and the equivalent terms such as “soluble film,” “orodispersible film,” “oral thin film,” “OTF,” “oral film,” “edible film,” “mucoadhesive film,” “mucoadhesive oral film,” “MOF,” “oral disintegrating film,” “oral soluble film,” “OSF,” etc.) refers to a soluble film specifically configured for oral administration. The term also includes “buccal film” (oral dissolvable film intended to be placed in the buccal space) and “sublingual film” (oral dissolvable film intended to be placed under the tongue). The oral dissolvable film is self-supporting, or in other words, is able to maintain its integrity and structure in the absence of a separate support. Oral dissolvable films are composed of pharmaceutically acceptable ingredients that are edible or ingestible. The oral dissolvable film can be configured for multi- or unidirectional release. ODFs can be similar in size and shape to a postage stamp, and are designed for oral administration, with the user placing the strip on the tongue (enteric), under the tongue (sublingual), through the oral mucosa (mucosal), against the inside of the cheek (buccal), or on the gums (gingival). Aside from the enteric route, these drug delivery options allow the medication to bypass the first pass metabolism thereby making the medication more bioavailable. As the film dissolves, the drug can enter the blood stream enterically, mucosally, buccally, gingivally, and/or sublingually. As such, the oral dissolvable film is prepared using hydrophilic polymers that can dissolve on the tongue or buccal cavity, delivering the drug to the systemic circulation via dissolution when contact with liquid is made. Oral film drug delivery accordingly uses a dissolving film to administer drugs via absorption in the mouth (buccally, sublingually, or gingivally) and/or via the small intestines (enterically). Especially for drugs which are metabolized extensively by the first-pass effect, oral films described herein can provide a faster-acting and better absorption profile. The oral dissolvable film can be shaped as being circular, round, oval, elliptical, or polygonal (e.g., triangular, square, or rectangular). Additionally, the ODF can optionally include a logo and/or indicia located thereon. The logo and/or indicia can identify, e,g., the marketing company name, manufacturing company name, drug substance name, drug product name, strength, dosage form, route of administration, and/or product serialization. Such logo and/or indicia can be printed thereon, e.g., with pharmaceutically acceptable ink, or can be embossed.

The term “pore” refers to a hole, aperture, channel, crack, cavity, perforation, cleft, crannied, fissure, gap, crevice, incision, recess, socket, opening, groove, pocket, or slit. The pore can be introduced in the cast slurry or the cured film. When present therein, the pore can extend completely through the cast slurry and/or cured film. Alternatively, the pore can extend only partially through the cast slurry and/or cured film. The pore can have any suitable size, shape and can form any suitable pattern in the cast slurry and/or cured film. Additionally, the geometry or pattern of the pores can be substantially uniform or non-uniform. Either way, the presence of the pores in an oral dissolvable film provide for an increased surface area relative to the equivalent oral dissolvable film not having the pores but having the same mass. An oral dissolvable film with an increased surface area will typically have a quicker dissolution and/or disintegration. As such, when placed in the oral cavity, the oral dissolvable film containing the pores (i.e., having an increased surface area) will typically have a quicker dissolution and/or disintegration, relative to the equivalent oral dissolvable film not having the pores, but having the same mass.

The term “honeycomb,” “honeycomb pattern,” or “honeycomb geometry” refers to a pattern consisting of an array of pores of substantially the same size, in which the axes of the pores are quasihorizontal and the nonangled rows of the pores are horizontally (not vertically) aligned. The geometry or pattern will typically be substantially uniform. The term honeycomb does not mean that the shape of the individual pores necessarily be hexagonal. While the pores may have a polygonal (e.g., hexagonal) shape, they typically will have a substantially round shape (e.g., oval, circular, or elliptical). With a honeycomb pattern, any given line of pores will have multiple perpendicular (and intersecting) lines of pores, as well as multiple parallel lines of pores.

The term “linear pattern” or “crisscross pattern” refers to a pattern consisting of an array of pores of substantially the same size, in which the axes of the pores are aligned parallel and/or perpendicular to one another. As such, any one axes of the linear pattern will have multiple parallel axes as well as multiple perpendicular (and intersecting) axes. The geometry or pattern will typically be substantially uniform. With a linear pattern, any given line of pores will have multiple perpendicular (and intersecting) lines of pores, as well as multiple parallel lines of pores.

The term “anhydrous film” refers to an oral dissolvable film containing no significant or appreciable amount of water. As such, reference to the weight percentage amount of water (moisture) present in an anhydrous film is essentially zero. Within the context of the present invention, it is appreciated that those of skill in the art understand and agree that an oral dissolvable film will likely include at least trace amounts of water as the complete removal of the water during the curing phase is unlikely. And that the oral dissolvable film may pick up moisture during the packaging, shipment, and/or storage. However, with the exception of the solvent water, substances (e.g., excipients and API) present in the orally soluble film described herein can be characterized by the weight percentage amount, based on an anhydrous film. As such, within the context of the present invention, it is appreciated that those of skill in the art understand and agree that reference to the oral dissolvable film as being anhydrous, at least for purposes of expressing the weight percentage amount of the excipients and/or API (with the notable exception of the solvent water) is otherwise acceptable and appropriate.

The term “hydrated film” refers to an oral dissolvable film containing a significant and appreciable amount of water. The amount of water present in a hydrated film can be measured (e.g., loss on drying).

The term “unit dosage” (and equivalent terms such as “unit dose” and “unit dosage form”) refers to an oral dissolvable film sized to the appropriate dimension, such that the individual film contains a desired amount of active ingredient to be administered to a subject for an intended use. Prior to sizing to the appropriate dimension (thereby providing the unit dosage form), the soluble film can exist, e.g., in either the unwound form (e.g., sheet) or in the wound form (e.g., bulk roll).

The term “drug substance” refers to the unformulated API (active pharmaceutical ingredient). The API has the therapeutic effect in the body as opposed to the excipients, which assist with the delivery of the API. The chemical purity and physical state (crystal form, salt form, etc.) can influence the quality and performance of a drug substance. This is especially important for water insoluble drugs.

The term “drug product” refers to the formulated drug substance with excipients. The drug product is typically the final marketed dosage form of the drug substance, for example a tablet, capsule, or oral dissolvable film (ODF). A drug substance, because of multiple factors (sensitivity, stability, etc.) is often mixed with other components before being released for use in the market. The drug substance together with the added ingredients (excipients) is known as drug product. The drug substance together with these added agents is called the drug product and within its packaging is called the “finished product.”

The term “drug load” (and equivalent terms such as “load of active ingredient”) refers to the amount of active pharmaceutical ingredient present in the oral dissolvable film. For example, in specific embodiments the oral dissolvable film can have a high drug load, such that the active pharmaceutical ingredient is present in a relatively high amount (e.g., above 30 wt. %) of the oral dissolvable film. The drug load is expressed as

$$\frac{weight of API}{weight of ODF} .$$

The term “treating” (and equivalent terms such as “treat,” “treated,” and “treatment”) of a subject includes the administration of an active pharmaceutical ingredient (API), or a unit dosage form containing the same (e.g., oral dissolvable film), to a subject with the purpose of preventing, mitigating, curing, healing, alleviating, relieving, altering, remedying, ameliorating, improving, stabilizing or affecting a disease or disorder, or a symptom of the disease or disorder.

The term “kit” refers to a system for delivering an oral dissolvable film as described herein, from one location to another. Such delivery systems can include enclosures that allow for the storage, transport, and/or delivery of an oral dissolvable film as described herein and any accompanying materials. The enclosure can be, e.g., a box or a bag. The accompanying materials can include, e.g., label, reference material, prescribing information, supporting material, or a combination thereof. For example, the kit can include a single dose of the oral dissolvable film described herein, that is individually packaged and sealed with a primary packaging material. Alternatively, the kit can include multiple doses of the oral dissolvable film described herein, each of which is individually packaged and sealed with a primary packaging material, Additionally, the kit can include an enclosure containing (i) multiple doses of the oral dissolvable film described herein, each individually packaged and sealed with a primary packaging material, and (ii) prescribing information. The primary packaging material can include at least one of metalized polyester, cellophane, polypropylene, nylon, polyester, vinylidene chloride, vinyl chloride, polycarbonate, low-density polyethylene, high-density polyethylene, linear low-density polyethylene, ionomer, polyvinyl alcohol, ethylene/vinyl acetate copolymer, ethylene/acrylic acid copolymer, ethylene/ethyl acrylate copolymer, polystyrene, and aluminum foil. The primary packaging material can include a single layer of material, or can include multiple layers of material (wherein each layer can independently include the same or different material as the other layers). The primary packaging material forms a primary package that can (i) protect the oral dissolvable film from light, (ii) protect the oral dissolvable film from microbial contamination, (iii) be child resistant, (iv) be a barrier to moisture and vapor, (v) mitigate leachable into the oral dissolvable film, (vi) identifies a logo and/or includes printed indicia, or (vii) any combination thereof. The logo and/or printed indicia can identify, e.g., the marketing company name, manufacturing company name, drug substance name, drug product name, strength, dosage form, route of administration, and/or product serialization.

The term “prescribing information” (and the equivalent terms “product information,” “product labeling,” “package insert,” or “PI”) refers to information relevant to the drug product, that is generally drafted by the drug company and such information is approved by the FDA. It includes the details and directions healthcare providers need to prescribe the drug product properly. It is also the basis for how the drug company can advertise its drug product. The prescribing information includes such details about the drug product as: its chemical description; how it works; how it interacts with other drugs, supplements, foods, and beverages; what condition(s) or disease(s) it treats; who should not use the drug product; serious side effects, even if they occur rarely; commonly occurring side effects, even if they are not serious; and effects on specific groups of patients, such as children, pregnant women, or older adults and how to use it in these populations. Sometimes, the drug company drafts prescription drug information designed for patients that the FDA approves. These are often called “Patient Package Inserts,” “Patient Product Information,” or “PPIs.”

The term “drug company” refers to the one or more companies that (i) markets the drug product (or retains ownership rights to the same), (ii) manufactures the drug product (or retains ownership rights to the same), and/or (iii) is the NDA applicant holder. As such, the drug company can be a single company or can include separate, multiple companies.

The term “subject” refers to living organisms such as humans, dogs, cats, and other mammals. Administration of the active ingredient included in the oral dissolvable film described herein can be carried out at dosages and for periods of time effective for the treatment of the subject. In some embodiments, the subject is a human.

The term “transmucosal” refers to any route of administration via a mucosal membrane or mucosal surface. Examples include, but are not limited to, buccal, sublingual, nasal, vaginal, and rectal.

The term “buccal administration” refers to a topical route of administration by which a drug held or applied in the buccal area (in the cheek) diffuses through the oral mucosa (tissues which line the mouth) and enters directly into the bloodstream. Buccal administration may provide better bioavailability of some drugs and a more rapid onset of action compared to oral administration because the medication does not pass through the digestive system and thereby avoids first pass metabolism. in multiple instances, buccal administration has been found to avoid liver and GI toxicities.

The term “buccal space” (also termed the buccinator space) refers to a fascial space of the head and neck (sometimes also termed fascial tissue spaces or tissue spaces). It is a potential space in the cheek, and is paired on each side. The buccal space is superficial to the buccinator muscle and deep to the platysma muscle and the skin. The buccal space is part of the subcutaneous space, which is continuous from head to toe.

The term “oral mucosa” refers to the mucous membrane lining the inside of the mouth and consists of stratified squamous epithelium termed oral epithelium and an underlying connective tissue termed lamina propria. Oral mucosa can be divided into three main categories based on function and histology: (1) Masticatory mucosa, keratinized stratified squamous epithelium, found on the dorsum of the tongue, hard palate and attached gingiva; (2) Lining mucosa, nonkeratinized stratified squamous epithelium, found almost everywhere else in the oral cavity, including the: (a) Buccal mucosa refers to the inside lining of the cheeks and floor of the mouth and is part of the lining mucosa; (b) Labial mucosa refers to the inside lining of the lips and is part of the lining mucosa; and (c) Alveolar mucosa refers to the lining between the buccal and labial mucosae. It is a brighter red, smooth and shiny with many blood vessels, and is not connected to underlying tissue by rete pegs; and (3) Specialized mucosa, specifically in the regions of the taste buds on lingual papillae on the dorsal surface of the tongue that contains nerve endings for general sensory reception and taste perception.

The term “sublingual administration,” from the Latin for “under the tongue,” refers to the pharmacological route of administration by which substances diffuse into the blood through tissues under the tongue. When a drug comes in contact with the mucous membrane beneath the tongue, it is absorbed. Because the connective tissue beneath the epithelium contains a profusion of capillaries, the substance then diffuses into them and enters the venous circulation. In contrast, substances absorbed in the intestines are subject to first-pass metabolism in the liver before entering the general circulation. Sublingual administration has certain advantages over oral administration. Being more direct, it is often faster, and it ensures that the substance will risk degradation only by salivary enzymes before entering the bloodstream, whereas orally administered drugs must survive passage through the hostile environment of the gastrointestinal tract, which risks degrading them, by either stomach acid or bile, or by enzymes such as monoamine oxidase (MAO). Furthermore, after absorption from the gastrointestinal tract, such drugs must pass to the liver, where they may be extensively altered; this is known as the first pass effect of drug metabolism. Due to the digestive activity of the stomach and intestines, the oral route is unsuitable for certain substances.

The term “gingival administration” refers to the pharmacological route of administration by which substances diffuse into the blood through tissues in the gums. The gums or gingiva (plural: gingivae), consist of the mucosal tissue that lies over the mandible and maxilla inside the mouth.

The term “enteral administration” refers to a drug administration via the human gastrointestinal tract. Enteral administration involves the esophagus, stomach, and small and large intestines (i.e., the gastrointestinal tract). Methods of administration include oral and rectal. Enteral administration may be divided into three different categories, depending on the entrance point into the GI tract: oral (by mouth), gastric (through the stomach), and rectal (from the rectum). (Gastric introduction involves the use of a tube through the nasal passage (NG tube) or a tube in the belly leading directly to the stomach (PEG tube). Rectal administration usually involves rectal suppositories.) Enteral medications come in various forms, including, e.g., tablets to swallow, chew or dissolve in water; capsules and chewable capsules (with a coating that dissolves in the stomach or bowel to release the medication there), oral dissolvable films, time-release or sustained-release tablets and capsules (which release the medication gradually), osmotic delivery systems, powders or granules, and liquid medications or syrups.

The term “oral administration” or “PO” refers to a route of administration where a substance is taken through the mouth. Many medications are taken orally because they are intended to have a systemic effect, reaching different parts of the body via the bloodstream.

The term “moisture content” (and equivalent terms such as “water content”) refers to the quantity of water contained in an oral dissolvable film described herein. The moisture content can encompass bound water and unbound water. Water content is expressed as a ratio, which can range from 0 (completely dry) to the value of the soluble film's porosity at saturation. It can be given on a volumetric or mass (gravimetric) basis. Typically, the moisture content will be expressed as a weight percent (e.g., 4 wt. %). Water content can be directly measured using a drying oven. Other methods that determine water content of a sample include chemical titrations (for example the Karl Fischer titration), determining mass loss on heating (perhaps in the presence of an inert gas), or after freeze drying. The Dean-Stark method is also commonly used. Unless specified otherwise, the loss on drying (LOD) method can be employed to calculate the moisture content of a soluble film described herein.

The term “loss on drying” or “LOD” refers to the loss of weight expressed as percentage w/w resulting from water and/or volatile matter that can be driven off under specified conditions from an object (e.g., oral dissolvable film). In this technique, a sample of material (e.g., oral dissolvable film) is weighed, heated in an oven for an appropriate period, cooled in the dry atmosphere of a desiccator, and then reweighed. The difference in weight is the loss on drying (LOD). For example, the oral dissolvable film can have a loss on drying (LOD) of 5±2 wt. %. Methods employed include Thermogravimetric Analysis (TGA) and IR, Moisture Analyzers.

The term “pharmaceutically acceptable” refers to those compounds, excipients, active ingredients, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “disintegration” refers to a physical process of breaking down a substance into fragments to improve its solubility in a solvent. The process is used predominantly in pharmaceutical and chemical industries. Disintegration occurs when a dosage form breaks up into smaller particles. It usually takes place in two steps; the content breaks up into small granules which then disaggregate. For oral dissolvable films, this breaking up process of the dosage form usually starts in the oral cavity, where it may be completed depending upon, e.g., the formulation, contact duration, volume of saliva, and whether or not taken with a beverage. If the dosage form passes into the stomach and then into the small intestine, then the process may continue there. Disintegration can usually be observed in the laboratory in a dissolution apparatus. Actual QC disintegration methods, however, use specific pieces of equipment described in USP <701>.

The term “dissolution” refers to a process through which solutes dissolve in a solvent. Dissolution is used predominantly in pharmaceutical industries to check how soluble a drug is in the body. Dissolution is a process through which a dosage form dissolves in a solvent to produce a solution. Dissolution requires disintegration of the dosage form to occur first, then drug particles to dissolve. It is the rate (amount of drug and time) at which the drug dissolves. In vivo dissolution of an oral dissolvable film starts as soon as the drug in the dosage form is wetted with saliva. There are many different dissolution apparatuses used to establish how much drug dissolves and how long this takes. The standard apparatus is also described in USP <711>.

The term “bioavailability” refers to a subcategory of absorption and is the fraction (%) of an administered drug that reaches the systemic circulation. Mathematically, bioavailability equals the ratio of comparing the area under the plasma drug concentration curve versus time (AUC) for the extravascular formulation to the AUC for the intravascular formulation. AUC can be utilized because AUC is proportional to the dose that has entered the systemic circulation.

The term “curing” refers to a chemical process that can be used to produce a soluble film (as described herein) from a slurry (also described herein). The process can be carried out by removing solvent (water), by toughening or hardening of polymer material present in the slurry, by cross-linking the polymer chains, etc. The term curing can be used to refer to the processes where starting from a liquid or semi-solid solution (e.g., slurry), a solid product soluble film) is obtained. Curing can be initiated by heat, radiation, electron beams, or chemical additives. To quote from IUPAC: curing “might or might not require mixing with a chemical curing agent.” IUPAC. Compendium of Chemical Terminology, 2nd ed. (the “Gold Book”). Compiled by A. D. McNaught and A. Wilkinson. Blackwell Scientific Publications, Oxford (l997). Online version (2019-) created by S. J. Chalk. ISBN 0-9678550-9-8. https://doi.org/10.1351/goldbook. Thus, two broad classes are (i) curing induced by chemical additives (also called curing agents, hardeners) and (ii) curing in the absence of additives. An intermediate case involves a mixture of resin and additives that requires external stimulus (light, heat, radiation) to induce curing.

The term “mass” refers to a measurement of how much matter is in an object. Mass is a combination of the total number of atoms, the density of the atoms, and the type of atoms in an object. Mass is usually measured in grams (which is abbreviated as g) or milligrams (which is abbreviated as mg).

The term “density” refers to the mass per unit volume of an object (e.g., oral dissolvable film). Density is calculated by dividing the mass of an object by the volume of the object. The volume of an object can be stated as cubic centimeters or milliliters as both are equivalent.

The term “tack” refers to the tenacity with which the oral dissolvable film adheres to an accessory (a piece of paper) that has been pressed into contact with the film.

The term “tensile strength” refers to the maximum stress applied to a point at which the oral dissolvable film specimen breaks. It is calculated by the applied load at rupture divided by the cross-sectional area of oral dissolvable film, as given in the equation below:

Tensile strength=Load at failure×100/Film thickness×Film width

The term “percent elongation” refers to the relative increase in amount in length upon application of stress. When stress is applied on a film sample, it gets stretched. This is referred to as strain. Strain is basically the deformation of film before it gets broken due to stress. it can be measured by using hounsfield universal testing machine. Generally, elongation of the film increases as the plasticizer content increases. It is calculated by the formula:

% Elongation=increase in length of film×100/Initial length of film

The term “tear resistance” refers to the resistance which a film offers when some load or force is applied on the film specimen. Specifically, it is the maximum force required to tear the specimen. The load mainly applied can be of a very low rate (e.g., 51 mm/min). The unit of tear resistance is Newton or pounds-force.

The term “Young's modulus” (and equivalent terms such as “elastic modulus” refers to the measure of stiffness of a soluble film. It is represented as the ratio of applied stress over strain in the region of elastic deformation as follows:

Young's modulus=Slope×100/Film thickness/Cross head speed

Hard and brittle strips demonstrate a high tensile strength and Young's modulus with small elongation.

The term “folding endurance” refers to number of times the film can be folded without breaking or without any visible crack. Folding endurance is a measure of the brittleness of a film. The method followed to determine endurance value is that the film specimen is repeatedly folded at the same place until it breaks, or a visible crack is observed. The number of times the film is folded without breaking or without any visible crack is the calculated folding endurance value.

The term “drug content uniformity” (and equivalent terms such as “uniformity of dosage unit” or “CU”) refers to the degree of uniformity in the amount of drug substance among dosage units, and unless otherwise specified, is set forth in USP-NF General Chapter <905> Uniformity of Dosage Units.

### Manufacturing, Packaging, and Distribution

The manufacture of oral dissolvable films can be carried out by various methods such as: (1) casting (e.g., solvent casting or semi-solid casting), (2) extrusion (e.g., hot melt extrusion or solid dispersion), and (3) rolling. These methods of manufacturing oral dissolvable films are generally well-known to the skilled artisans. See, e.g., “Manufacturing Techniques of Orally Dissolving Films,” Pharmaceutical Technology, Volume 35, issue 1 (Jan. 2, 2011); “Current Advances in Drug Delivery Through Fast Dissolving/Disintegrating Dosage Forms,” Vikas Anand Saharan, pp. 318-356 (39) (2017); A short review on “A novel approach in oral fast dissolving drug delivery system and their patents,” M. N. Siddiqui, G. Garg, P. K. Sharma, Adv. Biol. Res., 5 (2011), pp. 291-303; “Orally disintegrating films: A modern expansion in drug delivery system,” Ifran et al., Sandi Pharmaceutical Journal, Volume 24, Issue 5, pp. 537-546 (September 2016); “Development and characterization of pharmacokinetic parameters of fast-dissolving films containing levocetirizine,” D. R. Choudhary, V. A. Patel, U. K. Chhalotiya, H. V. Patel, A. J. Kundawala; Set. Pharm., 80 (2012), pp. 779-787; “Orally disintegrating preparations: recent advancement in formulation and technology,” R. R. Thakur, D. S. Rathore, S. Narwal; J. Drug Deliv. Therap., 2 (3) (2012), pp. 87-96; “Development of innovative orally fast disintegrating film dosage forms: a review,” B. P. Panda, N. S. Dey, M. E. B. Rao; Int. J. Pharm. Sci. Nanotechnol., 5 (2012), pp. 1666-1674.

Several methods for manufacturing an oral dissolvable film may be pursued, but the most common is solvent casting. Using this method, the manufacturing process can start with dispensing the excipients, active ingredient and solvent(s) in a defined order, preferably into a temperature-controlled tank and blending them into a slurry, typically using a high shear mixer to achieve a hoinogenous slurry. Homogeneity of the slurry should be tested by sampling at different locations in the tank and measuring viscosity and solids content. Depending on the properties of the slurry (i.e., bacteriostatic, bactericidal or growth promoting), in-process bioburden testing may be employed. To ensure flexibility of production scheduling, optimal conditions for storing the slurry should be tested.

The slurry may then be fed into an oven through a coating station, typically using a pump system. The slurry may be applied to a liner using a slot die or knife-over-roll coater, at a determined pin gauge. The selection of the liner can affect how the solvent (e.g., water) will evaporate and should generally be selected as to mitigate the occurrence of any chemical interaction with the film. Relevant parameters when selecting a liner are moisture content of the cast film, the location of the heat source and the directionality and strength of the air flow within the oven. It is generally preferred to “bake” the film rather than “broil” it. Additionally, the liner will preferably be qualified by the FDA, e.g., having its own drug master file (DMF).

Casting parameters—oven temperature, pin gauge and belt speed—required to meet product specifications at the terminal end of the oven, are generally optimized to achieve the fastest belt speed for highest throughput and cost efficiencies. Some oven systems enable the operator to control the height and directionality of the air nozzles and offer modular heat zones (e.g., infrared, progressive temperature increase). Oven lengths generally range from 10 ft to 24 ft, or more. The film and liner may then be packed as master rolls at the terminal end of the oven and should be stored in a temperature and humidity-controlled. environment as oral dissolvable films tend to be hygroscopic. Stability of the intermediate master rolls over time may be established by evaluating API content, moisture levels, physical characteristics, pliability dissolution, microbiology, and tensile strength. Typically, oral dissolvable film products are stable at room temperature in an appropriate container closure system. Of note, there is currently no official method or monograph in the US Pharmacopeia for evaluating oral dissolvable film properties such as disintegration, dissolution or mucoadhesion. Additionally, at the end of casting, additional API and/or occluding layer can be sprayed onto the cast film. Alternatively, the film can be 3D printed instead of casting.

Another step in the production is cutting the master roll into daughter rolls (alternatively referred to as “sitting”) and further into single doses which can be placed into individual pouches or sachets by converting and packaging machines. Ink priritirig of a mark on the film can be carried out. The mark can include a logo or indicia, such as the company name and dosage. The ink is preferably pharmaceutically acceptable and will not interfere with the performance characteristics (e.g., disintegration time) of the ODF. The API dose of an oral dissolvable film product is directly informed by the weight of the film. It is therefore critical to control the weight of each film product that is packaged. The size to which each individual film should cut must be determined during process development to ensure the product meets its the target weight and API load. A significant advantage with this dosage form is the ease in which multiple stock keeping units (SKUs) can be produced, by simply modifying the size or mass of the film.

Metalized polyester is a suitable primary packaging material for oral dissolvable film. It can be cost effective and protects the product from moisture and light. This pouch material can be child resistant and closure systems can be designed to ensure the product passes child resistant testing, while remaining user friendly for the patient. The pouches or sachets can offer larger printable 2D areas which traditional drug product formats do not. This allows the manufacturer to adapt to rapidly evolving labeling and regulatory requirements for information and anti-counterfeiting, such as product serialization. Furthermore, the primary package can provide sufficient space to include instructions on how to open the package and use the product, so that patients have a clear understanding of how it works. If desirable, each ODF can be individually packaged. One supplier of commercially available primary packaging materials is Bemis Company, Inc. (Neenah, Wis.).

The manufacturing of oral dissolvable films can be a continuous but modular process that is suitable to automation. The modularity of the process, such as master roll holds, allows for finished conversion to be done in the country or region of distribution, which compliments satellite expansion based on regional demand. The manufacturing process can have a low carbon footprint, with lower use of water for component preparation, as compared with other dosage forms.

The term “laser” refers to a device that emits light through a process of optical amplification based on the stimulated emission of electromagnetic radiation. The word “laser” is an acronym for “light amplification by stimulated emission of radiation.”

The term “impregnated” (or “embedded”) refers to a substance (e.g., active pharmaceutical ingredient) being inserted or located in a substrate (e.g., pores of the cured film), such that the substrate becomes an integral part of a surrounding to the substance. For example, the API can be impregnated into the pores of the cured film, such that substantially all of the API is located within the pores of the cured film. Alternatively, the API can be impregnated into the pores of the cured film, such that a portion of the API is located within the pores of the cured film. Either way, the pores containing the API can be referred to as being impregnated with the API.

### Specific Ranges, Values, and Embodiments

The specific embodiments describing the ranges and values provided below are for illustration purposes only, and do not otherwise limit the scope of the disclosed subject matter, as defined by the claims.

In specific embodiments, the pores extend therethrough the oral dissolvable film.

In specific embodiments, the pores extend partially therethrough the oral dissolvable film.

In specific embodiments, the pores extend at least 10% therethrough the oral dissolvable film.

In specific embodiments, the pores extend at least 25% therethrough the oral dissolvable film.

In specific embodiments, the pores extend at least 50% therethrough the oral dissolvable film.

In specific embodiments, the pores extend at least 75% therethrough the oral dissolvable film.

In specific embodiments, the pores extend at least 90% therethrough the oral dissolvable film.

In specific embodiments, the pores extend completely therethrough the oral dissolvable film.

In specific embodiments, the oral dissolvable film does not include an active pharmaceutical ingredient (API).

In specific embodiments, the oral dissolvable film includes an active pharmaceutical ingredient (API).

In specific embodiments, the oral dissolvable film includes one or more active pharmaceutical ingredients (APIs).

In specific embodiments, the oral dissolvable film includes multiple active pharmaceutical ingredients (APIs).

In specific embodiments, the oral dissolvable film includes one or more active pharmaceutical ingredients (APIs) impregnated into the pores.

In specific embodiments, the oral dissolvable film includes one or more active pharmaceutical ingredients (APIs) impregnated into the pores by printing.

In specific embodiments, the film forming agent of the film matrix is ingestible.

In specific embodiments, the film forming agent of the film matrix is water-soluble, water swellable, or a combination thereof.

In specific embodiments, the one or more pharmaceutically acceptable excipients of the film matrix includes at least one of binder, filler, preservative, sweetening agent, solvent, co-solvent, plasticizer, flavoring agent, taste masking agent, colorant, anti-caking agent, coating agent, emulsifier, solubilizing agent, lipid, humectant, thickening agent, lubricant, adsorbent, suspending agent, disintegrating agent, permeation enhancer, saliva stimulating agent, release modifier, adjuvant, fragrance, surfactant, pH adjusting agent, buffering agent, stabilizer, and antioxidant.

In specific embodiments, the film matrix is bioerodible.

In specific embodiments, the film matrix is mucoadhesive.

In specific embodiments, the pores are present in up to 100,000 pores per square inch of the oral dissolvable film.

In specific embodiments, the pores are present in up to 75,000 pores per square inch of the oral dissolvable film.

In specific embodiments, the pores are present in up to 50,000 pores per square inch of the oral dissolvable film.

In specific embodiments, the pores are present in up to 25,000 pores per square inch of the oral dissolvable film.

In specific embodiments, the pores are present in up to 10,000 pores per square inch of the oral dissolvable film.

In specific embodiments, the pores are present in at least 100 pores per square inch of the or a dissolvable film.

In specific embodiments, the pores are present in at least 250 pores per square inch of the oral dissolvable film.

In specific embodiments, the pores are present in at least 500 pores per square inch of the oral dissolvable film.

In specific embodiments, the pores are present in at least 750 pores per square inch of the oral dissolvable film.

In specific embodiments, the pores are present in at least 1,000 pores per square inch of the oral dissolvable film.

In specific embodiments, the pores are present in 100 to 100,000 pores per square inch of the oral dissolvable film.

In specific embodiments, the pores are present in 100 to 100,000 pores per square inch of the oral dissolvable film.

In specific embodiments, the pores are present in 250 to 100,000 pores per square inch of the oral dissolvable film.

In specific embodiments, the pores are present in 500 to 100,000 pores per square inch of the oral dissolvable film.

In specific embodiments, the pores are present in 750 to 100,000 pores per square inch of the oral dissolvable film.

In specific embodiments, the pores are present in 1,000 to 100,000 pores per square inch of the oral dissolvable film.

In specific embodiments, the pores are present in 100 to 75,000 pores per square inch of the oral dissolvable film.

In specific embodiments, the pores are present in 100 to 50,000 pores per square inch of the oral dissolvable film.

In specific embodiments, the pores are present in 100 to 25,000 pores per square inch of the oral dissolvable film.

In specific embodiments, the pores are present in 100 to 10,000 pores per square inch of the oral dissolvable film.

In specific embodiments, the pores are present in 250 to 75,000 pores per square inch of the oral dissolvable film.

In specific embodiments, the pores are present in 500 to 50,000 pores per square inch of the oral dissolvable film.

In specific embodiments, the pores are present in 750 to 25,000 pores per square inch of the oral dissolvable film.

In specific embodiments, the pores are present in 1,000 to 10,000 pores per square inch of the oral dissolvable film.

In specific embodiments, the oral dissolvable film is microporous, such that the pores have an average diameter of greater than 50 nm.

In specific embodiments, the oral dissolvable film is microporous, such that the pores have an average diameter of less than 50 nm.

In specific embodiments, the oral dissolvable film is microporous, such that the pores have an average diameter of less than 40 nm.

In specific embodiments, the oral dissolvable film is microporous, such that the pores have an average diameter of less than 30 nm.

In specific embodiments, the oral dissolvable film is microporous, such that the pores have an average diameter of less than 20 nm.

In specific embodiments, the oral dissolvable film is microporous, such that the pores have an average diameter of less than 10 nm.

In specific embodiments, the oral dissolvable film is microporous, such that the pores have an average diameter of greater than 2 nm.

In specific embodiments, the oral dissolvable film is microporous, such that the pores have an average diameter of greater than 5 nm.

In specific embodiments, the oral dissolvable film is microporous, such that the pores have an average diameter of greater than 7 nm.

In specific embodiments, the oral dissolvable film is microporous, such that the pores have an average diameter of greater than 10 nm.

In specific embodiments, the oral dissolvable film is microporous, such that the pores have an average diameter of 2 nm to 50 nm.

In specific embodiments, the oral dissolvable film is microporous, such that the pores have an average diameter of 5 nm to 25 nm.

In specific embodiments, the pores have an average diameter of at least 2 μm.

In specific embodiments, the pores have an average diameter of at least 5 μm.

In specific embodiments, the pores have an average diameter of at least 10 μm.

In specific embodiments, the pores have an average diameter of at least 25 μm.

In specific embodiments, the pores have an average diameter of at least 50 μm.

In specific embodiments, the pores have an average diameter of at least 75 μm.

In specific embodiments, the pores have an average diameter of at least 100 μm.

In specific embodiments, the pores have an average diameter of at least 200 μm.

In specific embodiments, the pores have an average diameter of less than 1,000 μm.

In specific embodiments, the pores have an average diameter of less than 750 μm.

In specific embodiments, the pores have an average diameter of less than 500 μm,

In specific embodiments, the pores have an average diameter of less than 250 μm.

In specific embodiments, the pores have an average diameter of 2 μm to 1,000 μm.

In specific embodiments, the pores have an average diameter of 2 μm to 1,000 μm.

In specific embodiments, the pores have an average diameter of 5 μm to 900 μm.

In specific embodiments, the pores have an average diameter of 10 μm to 800 μm.

In specific embodiments, the pores have an average diameter of 25 μm to 700 μm.

In specific embodiments, the pores have an average diameter of 50 μm to 600 μm.

In specific embodiments, the pores have an average diameter of 75 μm to 500 μm.

In specific embodiments, the pores have an average diameter of 100 μm to 400 μm.

In specific embodiments, the pores have an average diameter of 200 μm to 300 μm.

In specific embodiments, the aggregate area of the pores is at least 1% of the total area of the oral dissolvable film.

In specific embodiments, the aggregate area of the pores is at least 5% of the total area of the oral dissolvable film.

In specific embodiments, the aggregate area of the pores is at least 10% of the total area of the oral dissolvable film.

In specific embodiments, the aggregate area of the pores is at least 25% of the total area of the oral dissolvable film.

In specific embodiments, the aggregate area of the pores is at least 50% of the total area of the oral dissolvable film.

In specific embodiments, the aggregate area of the pores is at least 60% of the total area of the oral dissolvable film.

In specific embodiments, the aggregate area of the pores is less than 70% of the total area of the oral dissolvable film.

In specific embodiments, the aggregate area of the pores is less than 60% of the total area of the oral dissolvable film.

In specific embodiments, the aggregate area of the pores is less than 50% of the total area of the oral dissolvable film.

In specific embodiments, the aggregate area of the pores is less than 40% of the total area of the oral dissolvable film.

In specific embodiments, the aggregate area of the pores is less than 30% of the total area of the oral dissolvable film.

In specific embodiments, the aggregate area of the pores is less than 20% of the total area of the oral dissolvable film.

In specific embodiments, the aggregate area of the pores is less than 10% of the total area of the oral dissolvable film.

In specific embodiments, the aggregate area of the pores is less than 5% of the total area of the oral dissolvable film.

In specific embodiments, the aggregate area of the pores is 1% to 70% of the total area of the oral dissolvable film.

In specific embodiments, the aggregate area of the pores is 1% to 10% of the total area of the oral dissolvable film.

In specific embodiments, the aggregate area of the pores is 5% to 20% of the total area of the oral dissolvable film.

In specific embodiments, the aggregate area of the pores is 10% to 30% of the total area of the oral dissolvable film.

In specific embodiments, the aggregate area of the pores is 15% to 40% of the total area of the oral dissolvable film.

In specific embodiments, the aggregate area of the pores is 20% to 50% of the total area of the oral dissolvable film.

In specific embodiments, the aggregate area of the pores is 25% to 60% of the total area of the oral dissolvable film.

In specific embodiments, the aggregate area of the pores is 30% to 70% of the total area of the oral dissolvable film.

In specific embodiments, the pores are formed by mechanically piercing the oral dissolvable film.

In specific embodiments, the pores are formed by mechanically piercing the oral dissolvable film.

In specific embodiments, the pores are formed by mechanically pressing the oral dissolvable film.

In specific embodiments, the pores are formed by stretching the oral dissolvable film.

In specific embodiments, the pores are formed employing a rolling drum or microneedle arrays.

In specific embodiments, the pores are formed by contacting the cast slurry with particles of a substance that dissolve discrete portions of the cast slurry, and upon evaporation provide the pores.

In specific embodiments, the pores are formed by contacting the oral dissolvable film with particles of a substance that dissolve discrete portions of the oral dissolvable film, and upon evaporation provide the pores.

In specific embodiments, the pores are formed by laser light irradiation during curing.

In specific embodiments, the pores are formed by laser microarray drilling the oral dissolvable film after curing.

In specific embodiments, the pores are formed by a combination of (i) laser light irradiation during curing and (ii) by laser microarray drilling the oral dissolvable film after curing.

In specific embodiments, the pores are in the shape of a needle, cone, circle, oval, or slit.

In specific embodiments, the pores form a substantially uniform pattern or geometry on the oral dissolvable film.

In specific embodiments, the pores form a substantially uniform honeycomb (quasihorizontal) pattern or geometry on the oral dissolvable film.

In specific embodiments, the pores are in the form of a hole.

In specific embodiments, the pores are in the form of an aperture.

In specific embodiments, the pores are in the form of a channel.

In specific embodiments, the pores are in the form of a crack.

In specific embodiments, the pores are in the form of a cavity.

In specific embodiments, the pores are in the form of a perforation.

In specific embodiments, the pores are in the form of a cleft.

In specific embodiments, the pores are in the form of a cranny.

In specific embodiments, the pores are in the form of a fissure.

In specific embodiments, the pores are in the form of a gap.

In specific embodiments, the pores are in the form of a crevice.

In specific embodiments, the pores are in the form of an incision.

In specific embodiments, the pores are in the form of a recess.

In specific embodiments, the pores are in the form of a socket.

In specific embodiment e pores are in the form of an opening.

In specific embodiments, the pores are in the form of a groove.

In specific embodiments, the pores are in the form of a pocket.

In specific embodiments, the pores are in the form of a slit.

In specific embodiments, the oral dissolvable film is configured as a unit dosage form.

specific embodiments, the oral dissolvable film is configured as a self-supporting continuously cast film.

In specific embodiments, the film matrix is formed from a slurry which is a water-soluble film-forming matrix, water swellable film-forming matrix, or a combination thereof.

In specific embodiments, upon placing in the oral cavity, the oral dissolvable film disintegrates within 90 seconds.

In specific embodiments, upon placing in the oral cavity, the oral dissolvable film disintegrates within 75 seconds.

In specific embodiments, upon placing in the oral cavity, the oral dissolvable film disintegrates within 60 seconds.

In specific embodiments, upon placing in the oral cavity, the oral dissolvable film disintegrates within 45 seconds.

In specific embodiments, upon placing in the oral cavity, the oral dissolvable film disintegrates within 30 seconds.

In specific embodiments, upon placing in the oral cavity, the oral dissolvable film disintegrates within 20 seconds.

In specific embodiments, upon placing in the oral cavity, the oral dissolvable film disintegrates within 15 seconds.

In specific embodiments, the API is delivered sublingually, mucosally, enterally, buccally, or any combination thereof.

In specific embodiments, the API is delivered sublingually.

In specific embodiments, the API is delivered mucosally.

In specific embodiments, the API is delivered enterally.

In specific embodiments, the API is delivered buccally.

In specific embodiments, the oral dissolvable film has a thickness of 0.05 mm to 1.00 mm.

In specific embodiments, the oral dissolvable film has a thickness of at least 0.05 mm.

In specific embodiments, the oral dissolvable film has a thickness of at least 0.10 mm.

In specific embodiments, the oral dissolvable film has a thickness of at least 0.25 mm.

In specific embodiments, the oral dissolvable film has a thickness of at least 0.50 mm.

In specific embodiments, the oral dissolvable film has a thickness of at least 0.75 mm.

In specific embodiments, the oral dissolvable film has a thickness of less than 1.00 mm.

In specific embodiments, the oral dissolvable film has a thickness of less than 0.75 mm.

In specific embodiments, the oral dissolvable film has a thickness of less than 0.50 mm.

In specific embodiments, the oral dissolvable film has a thickness of less than 0.25 mm.

In specific embodiments, the oral dissolvable film has a thickness of less than 0.20 mm.

In specific embodiments, the oral dissolvable film is configured as a unit dosage form, having a mass of 180 mg to 260 mg.

In specific embodiments, the oral dissolvable film is configured as a unit dosage form, having a mass of at least 180 mg.

In specific embodiments, the oral dissolvable film is configured as a unit dosage form, having a mass of at least 190 mg.

In specific embodiments, the oral dissolvable film is configured as a unit dosage form, having a mass of at least 200 mg.

In specific embodiments, the oral dissolvable film is configured as a unit dosage form, having a mass of at least 210 mg.

In specific embodiments, the oral dissolvable film is configured as a unit dosage form, having a mass of less than 260 mg.

In specific embodiments, the oral dissolvable film is configured as a unit dosage form, having a mass of less than 250 mg.

In specific embodiments, the oral dissolvable film is configured as a unit dosage form, having a mass of less than 240 mg.

In specific embodiments, the oral dissolvable film is configured as a unit dosage form, having a mass of less than 230 mg.

In specific embodiments, the oral dissolvable film is configured as a unit dosage form, having a mass of less than 220 mg.

In specific embodiments, the oral dissolvable film is configured as a unit dosage form, having a mass of less than 210 mg.

In specific embodiments, the oral dissolvable film has a drug load of API of 0.5 wt. % to 40 wt. %.

In specific embodiments, the oral dissolvable film has a drug load of API of at least 0.5 wt. %.

In specific embodiments, the oral dissolvable film has a drug load of API of at least 1.0 wt. %.

In specific embodiments, the oral dissolvable film has a drug load of API of at least 5 wt. %.

In specific embodiments, the oral dissolvable film has a drug load of API of at least 10 wt. %.

In specific embodiments, the oral dissolvable film has a drug load of API of at least 15 wt. %.

In specific embodiments, the oral dissolvable film has a drug load of API of at least 20 wt. %.

In specific embodiments, the oral dissolvable film has a drug load of API of at least 25 wt. %.

In specific embodiments, the oral dissolvable film has a drug load of API of at least 30 wt. %.

In specific embodiments, the oral dissolvable film has a drug load of API of at least 35 wt. %.

In specific embodiments, the oral dissolvable film has a drug load of API of up to 40 wt. %.

In specific embodiments, the oral dissolvable film has a drug load of API of up to 35 wt. %.

In specific embodiments, the oral dissolvable film has a drug load of API of up to 30 wt. %.

In specific embodiments, the oral dissolvable film has a drug load of API of up to 25 wt. %.

In specific embodiments, the oral dissolvable film has a drug load of API of up to 20 wt. %.

In specific embodiments, the oral dissolvable film has a drug load of API of 1 wt. % to 40 wt. %.

In specific embodiments, the oral dissolvable film has a drug load of API of 5 wt. % to 40 wt. %.

In specific embodiments, the oral dissolvable film has a drug load of API of 10 wt. % to 40 wt. %.

In specific embodiments, the oral dissolvable film has a drug load of API of 15 wt. % to 40 wt. %

In specific embodiments, the oral dissolvable film has a drug load of API of 20 wt. % to 40 wt. %.

In specific embodiments, the oral dissolvable film has a drug load of API of 25 wt. % to 40 wt. %.

In specific embodiments, the oral dissolvable film has a density of 0.40 g;/cm³ to 0.90 g/cm³.

In specific embodiments, the oral dissolvable film has a density of at least 0.40 g/cm³.

In specific embodiments, the oral dissolvable film has a density of at least 0.45 g/cm³.

In specific embodiments, the oral dissolvable film has a density of at least 0.50 g/cm³.

In specific embodiments, the oral dissolvable film has a density of at least 0.55 g/cm³.

In specific embodiments, the oral dissolvable film has a density of at least 0.60 g/cm³.

In specific embodiments, the oral dissolvable film has a density of at least 0.65 g/cm³.

In specific embodiments, the oral dissolvable film has a density of at least 0.70 g/cm³.

In specific embodiments, the oral dissolvable film has a density of less than 0.90 g/cm³.

In specific embodiments, the oral dissolvable film has a density of less than 0.85 g/cm³.

In specific embodiments, the oral dissolvable film has a density of less than 0.80 g/cm³.

In specific embodiments, the oral dissolvable film has a density of less than 0.75 g/cm³.

In specific embodiments, the oral dissolvable film has a density of less than 0.70 g/cm³.

In specific embodiments, the oral dissolvable film has a density of less than 0.65 g/cm³.

In specific embodiments, the oral dissolvable film has a density of less than 0.60 g/cm³.

In specific embodiments, the oral dissolvable film has a density of 0.40 g/cm³ to 0.60 g/cm³.

In specific embodiments, the oral dissolvable film has a density of 0.50 g/cm³ to 0.70 g/cm³.

In specific embodiments, the oral dissolvable film has a density of 0.60 g/cm³ to 0.80 g/cm³.

In specific embodiments, the oral dissolvable film has a density of 0.70 g/cm³ to 0.90 g/cm³.

In specific embodiments, the oral dissolvable film has a dissolution (USP apparatus: 1 (basket); media: 0.01 N HCl; volume: 900-ml; temperature: 37±0.5° C.; RPM: 100; sampling: 5, 10, 15, 30, 45, 60 min) of at least 90 wt. % release at 5 minutes.

In specific embodiments, the oral dissolvable film has a dissolution (USP apparatus: 1 (basket); media: 0.01 N HCl; volume: 900-ml; temperature: 37±0.5° C.; RPM: 100; sampling: 5, 10, 15, 30, 45, 60 min) of at least 95 wt. % release at 5 minutes.

In specific embodiments, the oral dissolvable film has a dissolution (USP apparatus: 1 (basket); media: 0.01 N HCl; volume: 900-ml; temperature: 37±0.5° C.; RPM: 100; sampling: 5, 10, 15, 30, 45, 60 min) of at least 97.5 wt. % release at 5 minutes.

In specific embodiments, the oral dissolvable film has a dissolution (USP apparatus: 1 (basket); media: 0.01 N HCl; volume: 900-ml; temperature: 37±0.5° C.; RPM: 100; sampling: 5, 10, 15, 30, 45, 60 min) of 100 wt. % release at 5 minutes.

In specific embodiments, the oral dissolvable film includes a powder matrix located on at least one external surface of the oral dissolvable film, such that the powder matrix is in direct contact with the film matrix.

In specific embodiments, the oral dissolvable film includes a powder matrix located on at least one external surface of the oral dissolvable film, such that the powder matrix is in direct contact with the film matrix; wherein the powder matrix includes at least one of binder, filler, preservative, sweetening agent, solvent, co-solvent, plasticizer, flavoring agent, taste masking agent, colorant, anti-caking agent, coating agent, emulsifier, solubilizing agent, lipid, humectant, thickening agent, lubricant, adsorbent, suspending agent, disintegrating agent, permeation enhancer, saliva stimulating agent, release modifier, adjuvant, fragrance, surfactant, pH adjusting agent, buffering agent, stabilizer, and antioxidant.

In specific embodiments, the oral dissolvable film includes a single film matrix.

In specific embodiments, the oral dissolvable film includes multiple (es., 2, 3, 4, etc.) film matrices.

In specific embodiments, the oral dissolvable film includes multiple film matrices, wherein any one or more of the film matrices can independently be composed of the same substances present in the other film matrices.

In specific embodiments, the oral dissolvable film includes multiple film matrices, wherein any one of the matrices can independently be composed of different substances present in the other film matrices (e.g., non-uniform distribution of substances in the thickness direction among the multiple film matrices).

In specific embodiments, the oral dissolvable film includes multiple film matrices, wherein any one or more of the film matrices can independently include pores extending therethrough that film matrix (matrices), but not necessarily through the other film matrix (matrices).

In specific embodiments, the oral dissolvable film includes multiple film matrices, wherein any one or more of the film matrices can be engineered to independently include pores extending therethrough that film matrix (matrices), while the remaining film matrix (matrices) can be engineered to not include pores.

In specific embodiments, the method of manufacturing an oral dissolvable film containing pores extending therethrough includes (a) forming or obtaining a cast slurry, or forming or obtaining a cured film; and (b) laser irradiating multiple times the cast slurry or laser drilling multiple times the cured film, to form pores extending therethrough.

In specific embodiments, the method of manufacturing an oral dissolvable film containing pores extending therethrough includes (a) forming or obtaining a cast slurry, or forming or obtaining a cured film; (b) laser irradiating multiple times the cast slurry or laser drilling multiple times the cured film, to form pores extending therethrough; (c) converting cured film into desired dimensions; and (d) packaging.

In specific embodiments, the method of manufacturing an oral dissolvable film containing pores extending therethrough includes (a) forming or obtaining a cured film; (b) converting the cured film into desired dimensions; (c) laser irradiating multiple times the cured film or laser drilling multiple times the cured film, to form pores extending therethrough; and (d) packaging.

In specific embodiments, the method of manufacturing an oral dissolvable film containing pores extending therethrough includes (a) forming or obtaining a cast slurry, or forming or obtaining a cured film; (b) laser irradiating multiple times the cast slurry or laser drilling multiple times the cured film, to form pores extending therethrough; and (c) curing the cast slurry to form an oral dissolvable film with pores extending therethrough.

In specific embodiments, the method of manufacturing an oral dissolvable film containing pores extending therethrough includes (a) forming or obtaining a cured film; (b) converting the cured film into desired dimensions; (c) laser irradiating multiple times the cured film or laser drilling multiple times the cured film, to form pores extending therethrough; and (d) packaging.

In specific embodiments, the method of manufacturing an oral dissolvable film containing pores extending therethrough includes (a) forming or obtaining a cast slurry, or forming or obtaining a cured film; (b) laser irradiating multiple times the cast slurry or laser drilling multiple times the cured film, to form pores extending therethrough; (c) curing the cast slurry to form an oral dissolvable film with pores extending therethrough; (d) converting the cured film into desired dimensions; and (e) packaging.

In specific embodiments, the method of manufacturing an oral dissolvable film containing pores extending therethrough includes (a) forming or obtaining a cured film; (b) converting the cured film into desired dimensions; (c) laser irradiating multiple times the cured film or laser drilling multiple times the cured film, to form pores extending therethrough; and (d) packaging.

In specific embodiments, the method of manufacturing an oral dissolvable film containing pores extending therethrough includes (a) forming or obtaining a cured film; (b) laser drilling multiple times the cured film, to form pores extending therethrough; (c) converting the cured film into desired dimensions; and (d) packaging.

In specific embodiments, the method of manufacturing an oral dissolvable film containing pores extending therethrough includes (a) forming or obtaining a cured film; (b) converting the cured film into desired dimensions; (c) laser irradiating multiple times the cured film or laser drilling multiple times the cured film, to form pores extending therethrough; and (d) packaging.

In specific embodiments, the method of manufacturing an oral dissolvable film containing pores extending therethrough includes (a) forming or obtaining a cast slurry; (b) laser irradiating multiple times the cast slurry, to form pores extending therethrough; (c) curing the cast slurry to form an oral dissolvable film with pores extending therethrough; (d) converting the cured film into desired dimensions; and (e) packaging.

In specific embodiments, the method of manufacturing an oral dissolvable film containing pores extending therethrough includes (a) forming or obtaining a cured film; (b) converting the cured film into desired dimensions; (c) laser irradiating multiple times the cured film or laser drilling multiple times the cured film, to form pores extending therethrough; and (d) packaging.

In specific embodiments, the method of manufacturing an oral dissolvable film containing pores extending therethrough includes (a) forming or obtaining a cast slurry; (b) mechanically piercing the cast slurry, to form pores extending therethrough; and (c) curing the cast slurry to form an oral dissolvable film with pores extending therethrough.

In specific embodiments, the method of manufacturing an oral dissolvable film containing pores extending therethrough includes (a) forming or obtaining a cured film; and (b) mechanically piercing the cast slurry, to form pores extending therethrough.

In specific embodiments, the method of manufacturing an oral dissolvable film containing pores extending therethrough includes (a) forming or obtaining a cast slurry; (b) mechanically piercing the cast slurry, to form pores extending therethrough; (c) curing the cast slurry to form an oral dissolvable film with pores extending therethrough; (d) converting the cured film into desired dimensions; and (e) packaging.

In specific embodiments, the method of manufacturing an oral dissolvable film containing pores extending therethrough includes (a) forming or obtaining a cured film; (b) converting the cured film into desired dimensions; (c) mechanically piercing the cast slurry, to form pores extending therethrough; and (d) packaging.

In specific embodiments, the cured film in the forming or obtaining the cured film is formed by (c) forming or obtaining a slurry; (d) extruding the slurry and casting onto a substrate to form a cast slurry; and (e) curing the cast slurry to form a cured film.

In specific embodiments, the laser drilling or laser irradiating includes repeatedly pulsing focused laser energy on the cast slurry or cured film.

In specific embodiments, the laser drilling or laser irradiating is carried out at a rate of 0.3 pores per second to 3 pores per second.

In specific embodiments, the laser drilling or laser irradiating is carried out at a rate of at least 0.3 pores per second.

In specific embodiments, the laser drilling or laser irradiating is carried out at a rate of at least 0.5 pores per second.

In specific embodiments, the laser drilling or laser irradiating is carried out at a rate of at least 0,75 pores per second.

In specific embodiments, the laser drilling or laser irradiating is carried out at a rate of at least 1 pore per second.

In specific embodiments, the laser drilling or laser irradiating is carried out at a rate of at least 1.5 pores per second.

In specific embodiments, the laser drilling or laser irradiating is carried out at a rate of at least 2.0 pores per second.

In specific embodiments, the laser drilling or laser irradiating is carried out at a rate of at least 2.5 pores per second.

In specific embodiments, the laser drilling or laser irradiating is carried out at a rate of up to 3 pores per second.

In specific embodiments, the laser drilling or laser irradiating is carried out at a rate of up to 2.5 pores per second.

In specific embodiments, the laser drilling or laser irradiating is carried out at a rate of up to 2 pores per second.

In specific embodiments, the laser drilling or laser irradiating is carried out at a rate of up to 1.5 pores per second.

In specific embodiments, the laser drilling or laser irradiating is carried out at a rate of up to 1 pore per second.

In specific embodiments, the laser drilling or laser irradiating is carried out at a rate of 0.3 pores per second to 2.5 pores per second.

In specific embodiments, the laser drilling or laser irradiating is carried out at a rate of 0.3 pores per second to 3 pores per second.

In specific embodiments, the laser drilling or laser irradiating is carried out at a rate of 0.5 pores per second to 2 pores per second.

In specific embodiments, the laser drilling or laser irradiating is carried out at a rate of 0.3 pores per second to 3 pores per second.

In specific embodiments, the laser drilling or laser irradiating is carried out at a rate of 0.7 pores per second to 1.5 pores per second.

In specific embodiments, the laser drilling is carried out on the cured film, which is in the form of a self-supporting continuously cast film.

In specific embodiments, the laser drilling or laser irradiating is controlled with focal depth and intensity a linear rate of up to 0.2 ft²/s.

In specific embodiments, the laser drilling or laser irradiating is controlled with focal depth and intensity a linear rate of up to 0.15 ft²/s.

In specific embodiments, the laser drilling or laser irradiating is controlled with focal depth and intensity a linear rate of up to 0.1 ft²/s.

In specific embodiments, the laser drilling or laser irradiating is controlled with focal depth and intensity a linear rate of up to 0.05 ft²/s.

In specific embodiments, the laser drilling or laser irradiating is controlled with focal depth and intensity a linear rate of up to 0.01 ft²/s.

In specific embodiments, the laser includes a wavelength of 532 nm, pulse width of 3-5 ns, and repetition frequency of 1 Hz.

In specific embodiments, relative to an oral dissolvable film without the pores extending therethrough, the curing of the cast slurry to form an oral dissolvable film with pores extending therethrough is carried out over a shorter period of time and/or at a lower temperature.

In specific embodiments, the pores are formed by mechanically piercing the cast slurry.

In specific embodiments, the pores are formed by mechanically piercing the cured film.

In specific embodiments, the mechanical piercing is carried out employing a spiked wheel.

In specific embodiments, the pores are formed by mechanically piercing the cured film employing a spiked wheel.

In specific embodiments, the pores are formed by mechanically piercing the cast slurry employing a spiked wheel positioned between two ovens.

In specific embodiments, the pores can be formed by mechanically piercing the cast slurry employing a spiked wheel positioned between two ovens, so the cast slurry is sufficiently cured to withstand the holes but then goes through another zone of curing to complete the drying.

In specific embodiments, the pores facilitate the converting of the cured film into desired dimensions.

In specific embodiments, the pores facilitate sizing of the oral dissolvable film to a desired dimension.

In specific embodiments, the oral dissolvable film is scored with the pores, to facilitate sizing of the oral dissolvable film to a desired dimension.

In specific embodiments, the pores facilitate sizing of the oral dissolvable film to a desired dimension, such that the oral dissolvable film orally administered to the subject includes a desired dosage of active pharmaceutical ingredient(s).

In specific embodiments, the pores facilitate sizing of the oral dissolvable film to a desired dimension, such that the oral dissolvable film orally administered to the subject includes a titrated dosage of active pharmaceutical ingredient(s).

In specific embodiments, the pores facilitate sizing of the oral dissolvable film to a desired dimension, such that oral dissolvable film is in a unit dosage form containing multiple strengths of the active pharmaceutical ingredient(s).

In specific embodiments, the pores facilitate sizing of the oral dissolvable film, which is configured in a unit dosage form, to a desired dimension.

In specific embodiments, the pores facilitate sizing of the oral dissolvable film, which is configured in a unit dosage form, to a desired dimension, such that the oral dissolvable film orally administered to the subject includes a desired dosage of active pharmaceutical ingredient(s).

In specific embodiments, the pores facilitate sizing of the oral dissolvable film, which is configured in a unit dosage form, to a desired dimension, such that the oral dissolvable film orally administered to the subject includes a titrated dosage of active pharmaceutical ingredient(s).

In specific embodiments, the pores facilitate cutting or tearing of the oral dissolvable film, which is configured in a unit dosage form, to a desired dimension.

In specific embodiments, the pores facilitate cutting or tearing of the oral dissolvable film, which is configured in a unit dosage form, to a desired dimension, such that the oral dissolvable film orally administered to the subject includes a desired dosage of active pharmaceutical ingredient(s).

In specific embodiments, the pores facilitate cutting or tearing of the oral dissolvable film, which is configured in a unit dosage form, to a desired dimension, such that the oral dissolvable film orally administered to the subject includes a titrated dosage of active pharmaceutical ingredient(s).

In specific embodiments, the pores facilitate cutting or tearing of the oral dissolvable film, which is configured in a unit dosage form containing multiple strengths of the active pharmaceutical ingredient(s).

In specific embodiments, the pores are configured in a pattern with accompanying indicia.

In specific embodiments, the pores are configured in a pattern with accompanying indicia, proximally located to the pores.

In specific embodiments, the pores are configured in a pattern with accompanying indicia, proximally located to the pores to facilitate cutting or tearing of the oral dissolvable film.

### Enumerated Embodiments

Specific enumerated embodiments <1> to <61> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Embodiment 1

<1.> An oral dissolvable film including a film matrix, the film matrix including a film forming agent and one or more pharmaceutically acceptable excipients;
wherein,

the oral dissolvable film contains pores extending therethrough the oral dissolvable film.

### Embodiment 2

<2.> The oral dissolvable film of embodiment 1, further including one or more active pharmaceutical ingredients (APIs) impregnated into the pores.

### Embodiment 3

<3.> The oral dissolvable film of embodiment 1, further including one or more active pharmaceutical ingredients (APIs) impregnated into the pores by printing.

### Embodiment 4

<4.> An oral dissolvable film including a film matrix, the film matrix including a film forming agent, one or more active pharmaceutical ingredients (APIs), and one or more pharmaceutically acceptable excipients;
wherein,

the oral dissolvable film contains pores extending therethrough the oral dissolvable film.

### Embodiment 5

<5.> The oral dissolvable film of embodiment 4, including a single active pharmaceutical ingredient (API).

### Embodiment 6

<6.> The oral dissolvable film of embodiment 4, including multiple active pharmaceutical ingredients (APIs),

### Embodiment 7

<7.> The oral dissolvable film of any one of the above embodiments, wherein the oral dissolvable film contains pores extending partially therethrough the oral dissolvable film.

### Embodiment 8

<8.> The oral dissolvable film of any one of the above embodiments, wherein the oral dissolvable film contains pores extending completely therethrough the oral dissolvable film.

### Embodiment 9

<9.> The oral dissolvable film of any one of the above embodiments, wherein the film forming agent of the film matrix is ingestible.

### Embodiment 10

<10.> The oral dissolvable film of any one of the above embodiments, wherein the film forming agent of the film matrix is water-soluble, water swellable, or a combination thereof.

### Embodiment 11

<11.> The oral dissolvable film of any one of the above embodiments, wherein the film matrix is bioerodible.

### Embodiment 12

<12.> The oral dissolvable film of any one of the above embodiments, wherein the film matrix is mucoadhesive.

### Embodiment 13

<13.> The oral dissolvable film of any one of the above embodiments, wherein the one or more pharmaceutically acceptable excipients of the film matrix includes at least one of a binder, filler, preservative, sweetening agent, solvent, co-solvent, plasticizer, flavoring agent, taste masking agent, colorant, anti-caking agent, coating agent, emulsifier, solribilizing agent, lipid, humectant, thickening agent, lubricant, adsorbent, suspending agent, disintegrating agent, permeation enhancer, saliva stimulating agent, release modifier, adjuvant, fragrance, surfactant, pH adjusting agent, buffering agent, stabilizer, and antioxidant.

### Embodiment 14

<14.> The oral dissolvable film of any one of the above embodiments, wherein the pores are present in up to 100,000 pores per square inch of the oral dissolvable film.

### Embodiment 15

<15.> The oral dissolvable film of any one of the above embodiments, wherein the pores are present in 100 to 100,000 pores per square inch of the oral dissolvable film.

### Embodiment 16

<16.> The oral dissolvable film of any one of the above embodiments, which is a microporous material, such that the pores have an average diameter of less than 2 nm.

### Embodiment 17

<17.> The oral dissolvable film of any one of the above embodiments, which is a mesoporous material, such that the pores have an average diameter of 2 nm to 50 nm.

### Embodiment 18

<18,> The oral dissolvable film of any one of the above embodiments, which is a macroporous material, such that the pores have an average diameter of greater than 50 nm.

### Embodiment 19

<19.> The oral dissolvable film of any one of the above embodiments, wherein the pores have an average diameter of 2 μm to 1,000 μm.

### Embodiment 20

<20.> The oral dissolvable film of any one of the above embodiments, wherein the aggregate area of the pores is 1% to 70% of the total area of the oral dissolvable film.

### Embodiment 21

<21.> The oral dissolvable film of any one of the above embodiments, wherein the aggregate area of the pores is less than 5% of the total area of the oral dissolvable film.

### Embodiment 22

<22.> The oral dissolvable film of any one of the above embodiments, wherein the pores are formed by mechanically piercing the oral dissolvable film.

### Embodiment 23

<23.> The oral dissolvable film of any one of the above embodiments, wherein the pores are formed by stretching the oral dissolvable film. <Embodiment 24
<24.> The oral dissolvable film of any one of the above embodiments, wherein the pores are formed employing a rolling drum or microneedle arrays.

### Embodiment 25

<25.> The oral dissolvable film of any one of the above embodiments, wherein the pores are formed by:

contacting the cast slurry with particles of a substance that dissolve discrete portions of the cast slurry, and upon evaporation provides the pores; or

contacting the oral dissolvable film with particles of a substance that dissolve discrete portions of the oral dissolvable film, and upon evaporation provides the pores.

### Embodiment 26

<26.> The oral dissolvable film of any one of the above embodiments, wherein the pores are formed by laser light irradiation during curing.

### Embodiment 27

<27.> The oral dissolvable film of any one of the above embodiments, wherein the pores are formed by laser microarray drilling the oral dissolvable film after curing.

### Embodiment 28

<28.> The oral dissolvable film of any one of the above embodiments, wherein the pores are formed by a combination of (i) laser light irradiation during curing and (ii) by laser microarray drilling the oral dissolvable film after curing.

### Embodiment 29

<29.> The oral dissolvable film of any one of the above embodiments, wherein the pores are in the shape of a needle, cone, circle, oval, or slit.

### Embodiment 30

<30.> The oral dissolvable film of any one of the above embodiments, wherein the pores form a substantially uniform pattern or geometry on the oral dissolvable film.

### Embodiment 31

<31.> The oral dissolvable film of any one of the above embodiments, wherein the pores form a substantially uniform honeycomb (quasihorizontal) pattern or geometry on the oral dissolvable film.

### Embodiment 32

<32.> The oral dissolvable film of any one of the above embodiments, wherein the pores extend completely therethrough the oral dissolvable film.

### Embodiment 33

<33.> The oral dissolvable film of any one of the above embodiments, which is in a unit dosage form.

### Embodiment 34

<34.> The oral dissolvable film of any one of the above embodiments, which is in the form of a self-supporting continuously cast film.

### Embodiment 35

<35.> The oral dissolvable film of any one of the above embodiments, wherein the film matrix is formed from a slurry which is a water-soluble or water swellable film-forming matrix.

### Embodiment 36

<36.> The oral dissolvable film of any one of the above embodiments, wherein upon placing in the oral cavity, the oral dissolvable film disintegrates within 90 seconds.

### Embodiment 37

<37.> The oral dissolvable film of any one of the above embodiments, wherein the API is delivered sublingually, mucosally, enterally, buccally, or any combination thereof.

### Embodiment 38

<38.> The oral dissolvable film of any one of the above embodiments, having a thickness of 0.05 mm to 1.00 mm.

### Embodiment 39

<39.> The oral dissolvable film of any one of the above embodiments, which is in a unit dosage form and having a mass of 180-260 mg.

### Embodiment 40

<40.> The oral dissolvable film of any one of the above embodiments, having a drug load of API of 0.5 wt. % to 40 wt. %.

### Embodiment 4

<41.> The oral dissolvable film of any one of the above embodiments, having a density of 0.40 g/cm³ to 0.90 g/cm³.

### Embodiment 42

<42.> The oral dissolvable film of any one of the above embodiments, having a dissolution (USP apparatus: 1 (basket); media: 0.01 N HCl; volume: 900-ml; temperature: 37±0.5° C.; RPM: 100; sampling: 5, 10, 15, 30, 45, 60 min) of at least 90 wt. % release at 5 minutes.

### Embodiment 43

<43.> The oral dissolvable film of any one of the above embodiments, having a dissolution (USP apparatus: 1 (basket); media: 0.01 N HCl; volume: 900-ml; temperature: 37±0.5° C.; RPM: 100; sampling: 5, 10, 15, 30, 45, 60 min) of 100 wt. % release at 10 minutes.

### Embodiment 44

<44.> The oral dissolvable film of any one of the above embodiments, further including a powder matrix located on at least one external surface of the oral dissolvable film, such that the powder matrix is in direct contact with the film matrix.

### Embodiment 45

<45.> The oral dissolvable film of any one of the above embodiments, further including a powder matrix located on at least one external surface of the oral dissolvable film, such that the powder matrix is in direct contact with the film matrix; wherein the powder matrix includes at least one of a binder, filler, preservative, sweetening agent, solvent, co-solvent, plasticizer, flavoring agent, taste masking agent, colorant, anti-caking agent, coating agent, emulsifier, solubilizing agent, lipid, humectant, thickening agent, lubricant, adsorbent, suspending agent, disintegrating agent, permeation enhancer, saliva stimulating agent, release modifier, adjuvant, fragrance, surfactant, pH adjusting agent, buffering agent, stabilizer, and antioxidant.

### Embodiment 46

<46.> A method of manufacturing an oral dissolvable film containing pores extending therethrough, the method including:

(a) forming or obtaining a cast slurry, or forming or obtaining a cured film; and

(b) laser irradiating multiple times the cast slurry, laser drilling multiple times the cast slurry, laser drilling multiple times the cured film, or laser irradiating multiple times the cured film, to form pores extending therethrough.

### Embodiment 47

<47.> A method of manufacturing an oral dissolvable film containing pores extending therethrough, the method including:

(a) forming or obtaining a cast slurry, or forming or obtaining a cured film;

(b) laser irradiating multiple times the cast slurry, laser drilling multiple times the cast slurry, laser drilling multiple times the cured film, or laser irradiating multiple times the cured film, to form pores extending therethrough;

(c) converting the cured film into desired dimensions; and

(d) packaging.

### Embodiment 48

<48.> A method of manufacturing an oral dissolvable film containing pores extending therethrough, the method including:

(a) forming or obtaining a cast slurry, or forming or obtaining a cured film;

(b) converting the cured film into desired dimensions;

(c) laser irradiating multiple times the cast slurry, laser drilling multiple times the cast slurry, laser drilling multiple times the cured film, or laser irradiating multiple times the cured film, to form pores extending therethrough; and

(d) packaging.

### Embodiment 49

<49.> A method of manufacturing an oral dissolvable film containing pores extending therethrough, the method including:

(a) forming or obtaining a cast slurry;

(b) laser irradiating multiple times the cast slurry or laser drilling multiple times the cast slurry, to form pores extending therethrough; and

(c) curing the cast slurry to form an oral dissolvable film with pores extending therethrough.

### Embodiment 50

<50.> A method of manufacturing an oral dissolvable film containing pores extending therethrough, the method including:

(a) forming or obtaining a cast slurry;

(b) laser irradiating multiple times the cast slurry or laser drilling multiple times the cast slurry, to form pores extending therethrough;

(c) curing the cast slurry to form an oral dissolvable film with pores extending therethrough

(d) converting the cured film into desired dimensions; and

(e) packaging.

### Embodiment 51

<51.> A method of manufacturing an oral dissolvable film containing pores extending therethrough, the method including:

(a) forming or obtaining a cast slurry;

(b) curing the cast slurry to form an oral dissolvable film with pores extending therethrough

(c) converting the cured film into desired dimensions;

(d) laser irradiating multiple times the cast slurry or laser drilling multiple times the cast slurry, to form pores extending therethrough; and

(e) packaging.

### Embodiment 52

<52.> A method of manufacturing an oral dissolvable film containing pores extending therethrough, the method including:

(a) forming or obtaining a cured film;

(b) laser irradiating multiple times the cured film or laser drilling multiple times the cured film, to form pores extending therethrough;

(c) converting the cured film into desired dimensions; and

(d) packaging.

### Embodiment 53

<53.> A method of manufacturing an oral dissolvable film containing pores extending therethrough, the method including:

(a) forming or obtaining a cured film;

(b) converting the cured film into desired dimensions;

(c) laser irradiating multiple times the cured film or laser drilling multiple times the cured to form pores extending therethrough; and

(d) packaging.

### Embodiment 54

<54.> A method of manufacturing an oral dissolvable film containing pores extending therethrough, the method including:

(a) forming or obtaining a slurry;

(b) extruding the slurry and casting onto a substrate to form a cast slurry;

(c) curing the cast slurry to form a cured film;

(d) laser irradiating multiple times the cured film or laser drilling multiple times the cured film, to form pores extending therethrough;

(e) converting the cured film into desired dimensions; and

(f) packaging.

### Embodiment 55

<55.> A method of manufacturing an oral dissolvable film containing pores extending therethrough, the method including:

(a) forming or obtaining a slurry;

(b) extruding the slurry and casting onto a substrate to form a cast slurry;

(c) curing the cast slurry to form a cured film;

(d) converting the cured film into desired dimensions;

(e) laser irradiating multiple times the cured film or laser drilling multiple times the cured film, to form pores extending therethrough; and

(f) packaging.

### Embodiment 56

<56.> The method of any one of embodiments <46> to <55>, wherein the laser drilling or laser irradiating includes repeatedly pulsing focused laser energy on the cast slurry or the cured film.

### Embodiment 57

<57.> The method of any one of embodiments <46> to <56>, wherein the laser drilling or laser irradiating is carried out at a rate of 0.3 pores per second to 3 pores per second.

### Embodiment 58

<58.> The method of any one of embodiments <46> to <57>, wherein the laser drilling or laser irradiating is carried out on the cured film, which is in the form of a self-supporting continuously cast film.

### Embodiment 59

<59.> The method of any one of embodiments <46> to <58>, wherein the laser drilling or laser irradiating is controlled with focal depth and intensity a linear rate of up to 0.2 ft²/s. <Embodiment 60
<60.> The method of any one of embodiments <46> to <59>, wherein the laser includes a wavelength of 532 nm, pulse width of 3-5 ns, and repetition frequency of 1 Hz.

### Embodiment 61

<61.> The method of any one of embodiments <46> to <60>, that provides an oral dissolvable film of any one of embodiments <1> to <35>.

1. An oral dissolvable film comprising a film matrix, the film matrix comprising a film forming agent, one or more active pharmaceutical ingredients (APIs), and one or more pharmaceutically acceptable excipients;
wherein,
the oral dissolvable film contains pores extending therethrough;
upon placing in the oral cavity, the oral dissolvable film disintegrates within 90 seconds;
the oral dissolvable film has a thickness of 0.05 mm to 1.00 mm;
the oral dissolvable film is in a unit dosage form;
the oral dissolvable film has a mass of 180-260 mg;
the oral dissolvable film has a drug; load of API of 0.5 wt. % to 40 wt. %;
the oral dissolvable film has a density of 0.40 g/cm³ to 0.90 g/cm³.

2. The oral dissolvable film of claim 1, wherein the oral dissolvable film contains pores extending partially therethrough the oral dissolvable film.

3. The oral dissolvable film of claim 1, wherein the oral dissolvable film contains pores extending completely therethrough the oral dissolvable film.

4. The oral dissolvable film of claim 1, wherein the pores are in the form of a hole, aperture, channel, crack, cavity, perforation, cleft, cranny, fissure, gap, crevice, incision, recess, socket, opening, groove, pocket, or slit.

5. The oral dissolvable film of claim 1, comprising a single active pharmaceutical ingredient (API).

6. The oral dissolvable film of claim 1, wherein the film forming agent of the film matrix is ingestible.

7. The oral dissolvable film of claim 1, wherein the film forming agent of the film matrix is water-soluble, water swellable, or a combination thereof.

8. The oral dissolvable film of claim 1, wherein the film matrix is bioerodible.

9. The oral dissolvable film of claim 1, wherein the film matrix is mucoadhesive.

10. The oral dissolvable film of claim 1, wherein the one or more pharmaceutically acceptable excipients of the film matrix comprises at least one of a binder, filler, preservative, sweetening agent, solvent, co-solvent, plasticizer, flavoring agent, taste masking agent, colorant, anti-caking agent, coating agent, emulsifier, solubilizing agent, lipid, humectant, thickening agent, lubricant, adsorbent, suspending agent, disintegrating agent, permeation enhancer, saliva stimulating agent, release modifier, adjuvant, fragrance, surfactant, pH adjusting agent, buffering agent, stabilizer, and antioxidant.

11. The oral dissolvable film of claim 1, wherein the pores are present in at least 100 pores per square inch of the oral dissolvable film.

12. The oral dissolvable film of claim 1, wherein the pores are present in 100 to 100,000 pores per square inch of the oral dissolvable film.

13. The oral dissolvable film of claim 1, wherein the pores have an average diameter of at least 2 μm.

14. The oral dissolvable film of claim 1, wherein the pores have an average diameter of 2 μm to 1,000 μm.

15. The oral dissolvable film of claim 1, wherein the aggregate area of the pores is less than 5% of the total area of the oral dissolvable film.

16. The oral dissolvable film of claim 1, wherein the aggregate area of the pores is 0.1% to 5% of the total area of the oral dissolvable film.

17. The oral dissolvable film of claim 1, wherein the pores are mechanically formed.

18. The oral dissolvable film of claim 1, wherein the pores are chemically formed, by contacting the oral dissolvable film with particles of a substance that dissolve discrete portions of the oral dissolvable film, and upon evaporation provides the pores.

19. The oral dissolvable film of claim 1, wherein the pores are formed by (i) laser light irradiation, (ii) laser microarray drilling, or a combination thereof.

20. The oral dissolvable film of claim 1, wherein the pores are formed by laser light irradiation.

21. The oral dissolvable film of claim 1, wherein the pores are formed by laser microarray drilling.

22. The oral dissolvable film of claim 1, wherein the film matrix is formed from a slurry which is a water-soluble film-forming matrix, water swellable film-forming matrix, or combination thereof.

23. The oral dissolvable film of claim 1, wherein the API is delivered sublingually, mucosally, enterally, buccally, or any combination thereof.

24. The oral dissolvable film of claim having a dissolution (USP apparatus: 1 (basket); media: 0.01 N HCl; volume: 900-ml; temperature: 37±0.5° C.; RPM: 100; sampling: 5, 10, 15, 30, 45, 60 min) of at least 90 wt. % release at 5 minutes.

25. The oral dissolvable film of claim 1, further comprising a powder matrix located on at least one external surface of the oral dissolvable film, such that the powder matrix is in direct contact with the film matrix.
